SEQUENCE LISTING (3 sequences)

SEQ ID NO: 1 - protein, 114 aa, Artificial Sequence
  synthetic polypeptide
NWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 2 - protein, 115 aa, Artificial Sequence
  synthetic polypeptide
MNWVNVISDLKKIEDLIQSMHIDATLYTESDVHPSCKVTAMKCFLLELQVISLESGDASIHDTVENLIILANNSLSSNGNVTESGCKECEELEEKNIKEFLQSFVHIVQMFINTS

SEQ ID NO: 3 - protein, 56 aa, Artificial Sequence
  synthetic polypeptide
MRISKPHLRSISIQCYLCLLLNSHFLTEAGIHVFILGCFSAGLPKTEANWVNVISD

(19) United States
(12) Patent Application Publication
(10) Pub. No.: US 2023/0158164 A1
(43) Pub. Date: May 25, 2023

(54) METHOD FOR ENHANCING CELLULAR IMMUNOTHERAPY

(71) Applicants: Nektar Therapeutics, San Francisco, CA (US); Fred Hutchinson Cancer Research Center, Seattle, WA (US)

(72) Inventors: Antonio Mario Querido Marcondes, Millbrae, CA (US); Peter Benedict Kirk, Oxfordshire (GB); Takahiro Miyazaki, San Francisco, CA (US); Cameron J. Turtle, Seattle, WA (US); Stanley R. Riddell, Sammamish, WA (US); Cassie K. Chou, Seattle, WA (US); Simon P. Fräßle, Freising (DE)

(21) Appl. No.: 17/601,611

(22) PCT Filed: Apr. 4, 2020
(86) PCT No.: PCT/US2020/026778

§ 371 Date: Oct. 5, 2021

Related U.S. Application Data
(60) Provisional application No. 62/944,955, filed on Dec. 6, 2019.
(60) Provisional application No. 62/898,473, filed on Sep. 10, 2019.
(60) Provisional application No. 62/861,858, filed on Jun. 14, 2019.
(60) Provisional application No. 62/830,212, filed on Apr. 5, 2019.

(51) Int. Cl. A61K 47/64; A61K 35/17; C07K 16/28; A61P 35/00; A61K 9/00; A61K 38/17

(52) U.S. Cl. CPC A61K 47/642; A61K 35/17; C07K 16/2803; A61P 35/00; A61K 9/0019; A61K 38/1774; C07K 2317/24

(57) ABSTRACT

Provided are methods and compositions directed to the treatment of an individual having cancer by (i) administering to the individual an adoptive cellular immunotherapy composition comprising CAR T cells and (ii) administering to the individual an interleukin-15 receptor agonist, such as, for example, a long-acting interleukin-15 receptor agonist.

### CROSS REFERENCE TO RELATED APPLICATIONS

This application claims the benefit of priority under 35 U.S.C. § 119(e) to U.S. Provisional Patent Application No. 62/830,212, filed Apr. 5, 2019; U.S. Provisional Patent Application No. 62/861,858, filed Jun. 14, 2019; U.S. Provisional Patent Application No. 62/898,473, filed Sep. 10, 2019 and U.S. Provisional Patent Application No. 62/944,955, filed Dec. 6, 2019, the disclosures of which are each incorporated herein by reference.

### FIELD

The instant application relates to (among other things) the field of immunotherapy and involves the treatment of an individual having a condition such as cancer, by administering to the individual, chimeric antigen receptor (CAR) modified T cells and an interleukin-15 receptor agonist.

### BACKGROUND

New therapies are continually being developed for improved cancer treatment. One of the most promising areas of cancer treatment is immuno-oncology (i.e., cancer immunotherapy). Cancer immunotherapy refers to a diverse set of therapeutic strategies designed to modulate the immune response to induce a patient's own immune system to fight cancer. Among current immunotherapeutic approaches, adoptive cell transfer therapy (also referred to as ACT) has shown promise in treating patients with certain types of cancer. Adoptive cell therapy involves the isolation and ex vivo expansion of tumor specific lymphocytes to yield a greater number of tumor reactive effector T cells than could be achieved by simple vaccination. The tumor specific T cells are infused into patients with cancer to prime the patients' immune system to kill tumor cells. Adoptive cell transfer has shown effective clinical outcomes, particularly in metastatic melanoma (Dudley, M. E., J. R. Wunderlich, et al., J Clin Oncol 23(10): 2346-2357 (2005); Dudley, M. E., J. C. Yang, et al., J Clin Oncol 26(32): 5233-5239 (2008)). Adoptive cell transfer can be autologous, as is typical in adoptive T cell therapies, or can be allogeneic.

One form of adoptive T cell therapy is CAR T cell therapy (chimeric antigen receptor modified T cell therapy). CARs are a class of synthetic receptors that can reprogram lymphocyte specificity and function (Sadelain, M., et al., Nature, 545, 25 May 2017, p. 423-431). CAR T cell therapy uses ex vivo genetically engineered T cells transduced to express an artificial receptor which redirects the specificity of the T cells against a target tumor-associated antigen (TAA) expressed on the tumor cell surface (June, C. H., et al., Sci Transl Med 2015; 7(280):280 ps7). Unlike naturally-occurring T cells and T cell receptor engineered T cells that recognize their cognate antigen in the context of specific major histocompatibility complexes (MHC), antigen recognition by CAR T cells is MHC-independent, thereby expanding the applicability of this immunotherapeutic cell-based modality. First generation CAR T cells comprised the single chain variable region (scFv) of a monoclonal antibody, a T cell receptor transmembrane domain, and an intracellular signaling domain of CD3 zeta (CD3) chain. Later iterations also utilized one or more co-stimulatory domains, and/or a controllable on-off switch. CAR T cell therapies have advanced over time to provide genetically engineered T cells having improved specificities and safety profiles.

While CAR T cell based therapies have been approved in the U.S. for treating diffuse large B-cell lymphoma, not all patients respond to CAR T cells, and durability of response remains a limitation. An additional challenge for CAR T cell therapy is the poor survival of transferred cells. In relapsed or refractory large B-cell lymphoma, almost 60% of patients relapse or fail to progress, and the prognosis after failure is grim (Nair, J., et al., Best Practice & Research Clinical Haematology 31 (2018) 293-298). Successful CAR T cell therapy outcome, i.e., a favorable durable response with a complete remission rate at 6 months, is at least partially dependent upon long-term persistence of the CAR T cells. While clinical outcomes for Phase II trials of CAR T cell therapies in treating patients with lymphoma have reported notable efficacy, for many patients who initially respond to treatment, durability of response remains a limitation (Shah, N., et al., Frontiers in Oncology, 9 (March 2019) Art. 146). Additionally, acute toxicities that have been reported following CAR T cell therapy include cytokine release syndrome (CRS) and neurotoxicity referred to as CAR-related encephalopathy syndrome (Neelapu, S. S., et al., Nat Rev Clin Oncol 2018; 15(10:47-62). Other less commonly observed adverse side effects include hemophagocytic lymphohistiocytosis (HLH)/macrophage-activation syndrome (MAS), anaphylaxis, and tumor lysis syndrome.

Although there have been substantial efforts in developing effective CAR T cell based therapies to date, there remains a need to provide new and more effective immunotherapeutic CAR T cell strategies and related treatment regimens that address one or more of the disadvantages of current therapies.

Thus, the present disclosure seeks to address these disadvantages and other needs by providing a new and efficient CAR T cell-based immunotherapy that, for example, directs the CAR T cell population towards a tumor-killing phenotype having greater persistence and improved efficacy, among having other advantages (to be explained in greater detail below).

### SUMMARY

In a first aspect, provided herein is a method comprising adoptive cell transfer to a subject having cancer in combination with the administration of a long-acting IL-15 receptor agonist, to be described in greater detail herein. The present disclosure arises at least in part from the recognition that a combination treatment regimen including one or more cycles of adoptive cell therapy (e.g., by infusion of CAR T cells), and administration of a long acting IL-15 receptor agonist as described herein, administered sequentially, in either order, or administered substantially simultaneously, can be particularly effective in treating cancer in some subjects and/or can increase, enhance or prolong the activity and/or number of transferred cells, or result in a measurable beneficial response against cancer cells (e.g., stabilization, regression, shrinkage, necrosis, etc., as applicable), to an extent that is enhanced, preferably notably enhanced, over either single immunotherapeutic approach alone.

In a second aspect, provided herein is a combination immunotherapy for the treatment of a subject having cancer comprising administering to the subject an adoptive cellular immunotherapy composition comprising T cells that have been modified to express a chimeric antigen receptor (CAR T cells); and administering to the subject a long-acting IL-15 receptor agonist.

In a third aspect, provided is a method of improving the therapeutic effectiveness of adoptive cell therapy, such as for example, CAR T cell therapy, for treating a subject having cancer, the method comprising providing to a subject having cancer, an adoptive cellular immunotherapy composition comprising T cells that have been modified to express a chimeric antigen receptor; and administering to the subject a long-acting IL-15 receptor agonist, wherein administration of the long-acting IL-15 receptor agonist is effective to improve the subject's response to the adoptive cell therapy.

The following embodiments are meant to apply equally to each of the aspects described above, and to be considered both singly and in combination as applicable, unless indicated otherwise.

In some embodiments, the adoptive cell transfer comprises administering an adoptive cellular immunotherapy composition comprising T cells that have been modified to express a CD19-directed chimeric antigen receptor.

In one or more further embodiments, the long-acting IL-15 receptor agonist is effective to preferentially stimulate and expand natural killer (NK) cells. In yet one or more additional embodiments, the long-acting IL-15 receptor agonist supports CD8+ T cell survival and memory formation, e.g., without substantially inducing suppressive regulatory T cells (T regs). In yet some further embodiments, the long-acting IL-15 receptor agonist possesses IL-15 receptor alpha specificity. In some further embodiments, the long-acting IL-15 receptor agonist possesses one or more of the foregoing features, i.e., (i) is effective to preferentially stimulate and expand NK cells, (ii) supports CD8+ T cell survival and memory formation, e.g., without substantially inducing suppressive regulatory T cells (T regs), and (iii) possesses IL-15 receptor alpha specificity.

In some further embodiments, the long-acting IL-15 receptor agonist has a structure:

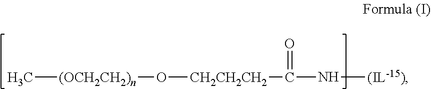

wherein IL-15 is an interleukin-15 moiety, (n) is an integer from about 150 to about 3,000, and ˜NH˜ represents an amino group of the IL-15 moiety.

In one or more embodiments related to the long-acting IL-15 receptor agonist of Formula (I), (n) is in a range from about 795 to about 1068. In some additional embodiments, (n) is in a range from about 840 to about 1023. In one or more particular embodiments, (n) has a value of, on average, about 907 or about 909.

By way of clarity, with regard to the sequence of administering, wherein the term “administering” is used in this instance to refer to delivery of either the adoptive cellular immunotherapy composition or the long acting IL-15 receptor agonist, the adoptive cells and the long acting IL-15 receptor agonist may be administered concurrently or sequentially and in any order. Moreover, treatment of either component of the combination may comprise a single cycle of therapy, or may comprise multiple cycles. That is to say, following an initial cycle of therapy comprising administration of the adoptive cellular immunotherapy composition comprising T cells that have been modified to express a chimeric antigen receptor, such as for example, CD19-directed CAR T cells, and administration of the long acting IL-15 agonist, additional rounds of therapy may include adoptive cell transfer, e.g., administration of CAR T cells in combination with administration of the long acting IL-15 receptor agonist, or adoptive cell therapy, e.g., CAR T cell therapy, that is not accompanied by administration of the long acting IL-15 receptor agonist, or administration of the long acting IL-15 receptor agonist that is not accompanied by adoptive cell transfer (e.g., administration of CAR T cells such as CD19 CAR T cells).

In one or more embodiments, the subject is a human subject.

In one or more non-limiting embodiments, the cancer is a liquid cancer such as a blood cancer, e.g., a relapsed or a refractory malignancy.

In one or more related non-limiting embodiments, the cancer is lymphoma or leukemia. In one or more related embodiments, the cancer is selected from Hodgkin and non-Hodgkin lymphoma.

In some additional non-limiting embodiments, the cancer is a B-cell malignancy. In some further embodiments, the cancer is B-cell lymphoma.

In yet some further embodiments, the cancer is multiple myeloma.

In one or more alternative embodiments, the cancer is a solid cancer.

In some further embodiments of a method as provided herein, the method results in a beneficial response to treatment that is enhanced over the response to treatment observed when administration is carried out according to administration of either the adoptive cellular immunotherapy composition or administration of the long-acting IL-15 receptor agonist alone.

In some embodiments related to the foregoing, a beneficial response to treatment is based on a suitable animal model, such as, for example, an in vivo xenogenetic B cell lymphoma model.

Additional aspects and embodiments are set forth in the following description and claims.

### BRIEF DESCRIPTION OF THE DRAWINGS

FIG. 1 provides the amino acid sequence of an exemplary recombinant human IL-15 from E. coli (SEQ ID NO:1); an exemplary recombinant human IL-15 including a methionine at the beginning of the sequence for initiation of translation in E. coli (SEQ ID NO:2), a single, non-glycosylated polypeptide chain containing 115 amino acids, with a molecular weight of 12.9 kDa; and an exemplary precursor form of IL-15 (SEQ ID NO: 3).

FIG. 2 is an illustration of a suitable CD19 CAR lentiviral construct used to transduce CD4 and CD8 T cells isolated from healthy donors as described in the Examples.

FIGS. 3A-3B illustrate the expression of IL-15Rα by human CD19 CAR T cells as measured by flow cytometry and further described in Example 2. Expression by CD8 CAR T cells is shown in FIG. 3A (solid line); IL-15Rα expression by CD4 CAR T cells is shown in FIG. 3B (solid line). Both figures also include an FMO control (filled gray), and an isotype control (dashed line).

FIGS. 3C-3D are graphs showing percent phosphorylation of STAT5 for CD8 CAR T cells (FIG. 3C) or CD4 CAR T cells (FIG. 3D) versus log of concentration (ng/mL) after stimulation with different concentrations of MPBA-IL15 (•) or IL-15 (▪) for 20 minutes as determined by flow cytometry, and as further described in Example 2.

FIGS. 3E-3F are graphs showing proliferation (% divided) of CD8 CAR T cells (FIG. 3E) and CD4 CAR T cells (FIG. 3F) labeled with CFSE and incubated with different concentrations of MPBA-IL15 (•) or IL-15 (▪) for 20 minutes as determined by flow cytometry, and as further described in Example 2.

FIG. 4A is a graph of average tumor radiance (p/s/cm²/sr) over time (days post CAR T cell infusion) as evaluated in an in vivo xenogeneic B cell lymphoma model as described in Example 3 for tumor-bearing mice treated with CAR T cells alone (▪), and in combination with different dosage amounts of mono(methoxyPEG-N-butanamide)interleukin-15 (MPBA-IL15): 0.030 mg/kg (green ♦), 0.10 mg/kg (purple ♦) and 0.30 mg/kg (▾). The PBS control is shown as circles (•).

FIG. 4B shows an illustrative treatment protocol for evaluating the efficacy of CD19 CAR T cell combination immunotherapy in a preclinical murine lymphoma model as described in Example 3 (Study A).

FIGS. 5A-5B are graphs showing the number of CD8 CAR T cells (FIG. 5A) or CD4 CAR T cells (FIG. 5B) in blood of lymphoma-cell bearing mice infused with CAR T cells (DO) followed by administration of MPBA-IL15 (0.3 mg/kg) starting on day −1, day 7 or day 14, and then weekly. Mice were bled weekly and CD8 and CD4 CAR T cells were identified by flow cytometry. Plots provide cell number/μl blood versus days post CAR T cell administration. Study groups included: tumor-bearing mice infused with MPBA-IL15 only (•), tumor-bearing mice infused with CAR T cells only (▪), and tumor bearing mice treated with CAR T cells in combination with MPBA-IL15 starting on day −1 (▴), day 7 (▾) or day 14 (♦) for up to 28 days post CAR T cell infusion as described in Example 3.

FIG. 6 is a graph assessing tumor burden by providing average tumor radiance (p/s/cm2/sr) over time (days post CAR T cell infusion) as evaluated in an in vivo xenogeneic B cell lymphoma model in tumor-bearing mice following infusion of MPBA-IL15 only (•), CAR T cells only (▪), or CAR T cells in combination with MPBA-IL15 starting on day−1 (▴), day 7 (▾) or day 14 (♦), and then weekly as described in Example 3.

FIG. 7A is a is a graph showing the percent survival of tumor-bearing mice treated with CAR T cells only (blue line), MPBA-IL15 only (black line), or CAR T cells in combination with MPBA-IL15 (0.3 mg/kg) starting on day −1 (purple line), day 7 (red line), or day 14 (green line) following infusion with the CAR T cells (day 0) as evaluated in an in vivo xenogeneic B cell lymphoma model as described in Example 3.

FIG. 7B shows an illustrative treatment protocol for evaluating the efficacy of CD19 CAR T cell combination immunotherapy in a preclinical murine lymphoma model as described in Example 3 (Study B).

FIGS. 8A-8C are plots of total numbers of CAR T cells, Ki67 expression, and PD1 and TIM3 expression, respectively, of CD8 CAR T cells from the bone marrow of Raji-lymphoma cell-bearing NSG mice infused with CAR T cells on DO (CAR T cell monotherapy) or having received, in addition to an infusion of CAR T cells, weekly injections of MPBA-IL15 starting on D7 as described in Example 3. FIG. 8A is a graph showing the total number of CD8 CAR T cells in the bone marrow of tumor-bearing mice following infusion of CAR T cells only (•), and in tumor bearing mice that received CAR T cell therapy in combination with MPBA-IL15 (▪). FIG. 8B is a graph showing Ki67 expression (% KI67+) in CD8 CAR T cells from the bone marrow of tumor-bearing mice following CAR T cell monotherapy (•), and from tumor-bearing mice that received CAR T cells in combination with MPBA-IL15 (▪). FIG. 8C is a graph showing PD1 and TIM3 expression (% PD1+TIM3+) in CD8 CAR T cells from the bone marrow of tumor-bearing mice following an infusion of CAR T cells only (•) (monotherapy), and from tumor-bearing mice that received CAR T cell therapy in combination with MPBA-IL15 (▪).

FIGS. 9A-9C are plots of total numbers of CAR T cells, Ki67 expression, and PD1 and TM3 expression, respectively, of CD4 CAR T cells from the bone marrow of Raji-lymphoma cell-bearing NSG mice infused with CAR T cells on DO (CAR T cell monotherapy) or having additionally received weekly injections of MPBA-IL15 starting on D7 as described in Example 3. FIG. 9A is a graph showing the total number of CD4 CAR T cells in the bone marrow of tumor-bearing mice following infusion of CAR T cells only (•), and in tumor bearing mice that received CAR T cell therapy in combination with MPBA-IL15 (▪). FIG. 9B is a graph showing Ki67 expression (% KI67+) in CD4 CAR T cells from the bone marrow of tumor-bearing mice following CAR T cell monotherapy (•), and from tumor-bearing mice that received CAR T cell therapy in combination with MPBA-IL15 (▪). FIG. 9C is a graph showing PD1 and TM3 expression (% PD1+TIM3+) in CD4 CAR T cells from the bone marrow of tumor-bearing mice following an infusion of CAR T cells only (•) (monotherapy), and from tumor-bearing mice that received CAR T cell therapy in combination with MPBA-IL15 (▪).

FIG. 10 provides bioluminescence images for representative timepoints for tumor-free mice previously treated with MPBA-IL-15 and CAR T cells, and rechallenged with Raji tumor cells on D38, followed by weekly imaging to assess tumor burden. This figure illustrates that mice previously treated with an exemplary long-acting IL-15 receptor agonist, MPBA-IL-15, and CAR T cells are able to reject Raji tumor rechallenge when evaluated in a mouse lymphoma model.

FIG. 11 shows an illustrative treatment protocol for evaluating the efficacy of ROR1 CAR T cell combination immunotherapy with an exemplary long-acting IL-15 receptor agonist, MPBA-IL15, in a preclinical murine lymphoma model as described in Example 4.

FIG. 12A is a graph of percent change in tumor volume for mice in the various treatment groups in the murine lymphoma model described in Example 4: control T cells (rectangles), control T cells and MPBA-IL15 (▴), ROR1 CAR T cells (▾), and ROR1 CAR T cells and MPBA-IL15 (♦); FIG. 12B is a graph of percent change in tumor volume versus weeks post-infection for individual mice treated with ROR1 CAR T cell monotherapy (18.5% regression for group)); FIG. 12C is a graph of percent change in tumor volume versus weeks post-infection for individual mice treated with ROR1 CAR T cell and MPBA-IL-15 doublet combination therapy (44.4% regression for group).

FIG. 13A is a plot of CD8 CAR T cell frequency expressed as a percentage of live cells in the spleen and tumor, respectively, for the treatment groups described in Example 4: control T cells (black), ROR1 CAR T cells (red), and ROR1 CAR T cells and MPBA-IL15 (blue); FIG. 13B is a plot of total CD8 cell frequency expressed as a percentage of live cells in the spleen and tumor, respectively, for the various treatment groups described in Example 4.

FIGS. 14A, 14B, 14C, and 14D are images of lung tissue from mice in various treatment groups in the ROR1 lung model illustrating that MPBA-IL-15, an illustrative long acting IL-15 agonist, augments ROR1 CAR T cell trafficking and persistence in the lung.

FIGS. 15A and 15B illustrate protein expression in CD8 CAR T cells following treatment with MPBA-IL15 in vitro as described in Example 5. FIG. 15A provides expression of IFNγ in pg/ml in CD8 CAR T cells co-cultured with irradiated K562-CD19+ or K562-CD19− cells, and either untreated with MPBA-IL15, or treated with MPBA-IL15 at concentrations of 1 ng/ml, 10 ng/ml, or 30 ng/ml. FIG. 15B provides expression of TNFα in pg/ml in CD8 CAR T cells co-cultured with irradiated K562-CD19+ or K562-CD19− cells, and either untreated with MPBA-IL15, or treated with MPBA-IL15 at concentrations of 1 ng/ml, 10 ng/ml, or 30 ng/ml.

FIG. 16A illustrates CAR T-cell expansion (as fold expansion) for CD8 CAR T cells co-cultured with irradiated K562-CD19+ or K562-CD19− cells, and either untreated with MPBA-IL15, or treated with MPBA-IL15 at concentrations of 1 ng/ml, 10 ng/ml, or 30 ng/ml as described in Example 5.

FIGS. 16B and 16C provide expression of bcl-2 (in bcl-2 MFI) and activated caspase 3 (as % caspase 3+), respectively, in CD8 CAR T cells co-cultured with irradiated K562-CD19+ or K562-CD19− cells, and either untreated with MPBA-IL15, or treated with MPBA-IL15 at concentrations of 1 ng/ml, 10 ng/ml, or 30 ng/ml as described in Example 5.

FIGS. 17A and 17B illustrate bcl-2 expression in CAR-T cells from lymphoma-bearing mice treated with CAR T cells either alone or in combination with MPBA-IL15 as described in detail in Example 6. Bcl-2 expression in CAR T cells is shown in histograms as determined on D8 post-infusion (grey=CAR T cell-only treated mice; red and blue=mice administered CAR T cells and mono(methoxyPEG-N-butanamide)interleukin-15 (MPBA-IL15)) for both CD8 CAR T cells (FIG. 17A) and CD4 CAR T cells (FIG. 17B).

FIGS. 18A and 18B also illustrate bcl-2 expression in CAR-T cells from lymphoma-bearing mice treated with CAR T cells either alone or in combination with MPBA-IL15, as described in detail in Example 6. Bcl-2 expression in CAR T cells is shown in bar graphs as determined on D8 post-infusion (black=CAR T cell-only mice; red=mice administered CAR T cells and mono(methoxyPEG-N-butanamide)interleukin-15 (MPBA-IL15)) for both CD8 CAR T cells (FIG. 18A) and CD4 CAR T cells (FIG. 18B).

FIGS. 19A-19D illustrate expression of memory markers CD45RA and CCR7 in CAR T cells as described in Example 6. FIG. 19A illustrates protein expression in CD8 CAR T cells of mice administered CAR T cells only; FIG. 19B illustrates protein expression in CD8 CAR T cells of mice administered CAR T cells and mono(methoxyPEG-N-butanamide)interleukin-15 (MPBA-IL15); FIG. 19C illustrates protein expression in CD4 CAR T cells of mice administered CAR T cells only; and FIG. 19D illustrates protein expression in CD4 CAR T cells of mice administered CAR T cells and mono(methoxyPEG-N-butanamide)interleukin-15 (MPBA-IL15), where expression data is provided for CD5RA−CCR7-(orange), CD5RA+CCR7− (green), and CD5RA−CCR7+(red). Graphs show mean±SEM.

### DETAILED DESCRIPTION

As used in this specification, the singular forms “a,” “an,” and “the” include plural referents unless the context clearly dictates otherwise.

In describing and claiming certain features of this disclosure, the following terminology will be used in accordance with the definitions described below unless indicated otherwise.

“PEG” or “polyethylene glycol,” as used herein, is meant to encompass any water-soluble poly(ethylene oxide). Unless otherwise indicated, a “PEG polymer” or a polyethylene glycol is one in which substantially all (preferably all) monomeric subunits are ethylene oxide subunits, though, the polymer may contain distinct end capping moieties or functional groups, e.g., for conjugation. PEG polymers for use in the present disclosure will comprise one of the two following structures: “—(CH₂CH₂O)ₙ—” or “—(CH₂CH₂O)ₙ₋₁CH₂CH₂—,” depending upon whether or not the terminal oxygen(s) has been displaced, e.g., during a synthetic transformation. For PEG polymers, the variable (n) typically ranges from about 3 to 4000, and terminal groups and architecture of the overall PEG can vary. Exemplary or preferred PEG-comprising molecules may however comprise one or more particular PEG architectures and/or linkers, and/or molecular weight ranges.

Molecular weight in the context of a water-soluble polymer, such as PEG, can be expressed as either a number (nominal) average molecular weight or a weight average molecular weight. Unless otherwise indicated, all references to molecular weight herein refer to the weight average molecular weight. Both molecular weight determinations, number average and weight average, can be measured using gel permeation chromatography or other liquid chromatography techniques (e.g. gel filtration chromatography). Most commonly employed are gel permeation chromatography and gel filtration chromatography. Other methods for determining molecular weight include end-group analysis or the measurement of colligative properties (e.g., freezing-point depression, boiling-point elevation, or osmotic pressure) to determine number average molecular weight or the use of light scattering techniques, ultracentrifugation, MALDI TOF, or viscometry to determine weight average molecular weight. PEG polymers are typically polydisperse (i.e., the number average molecular weight and the weight average molecular weight of the polymers are not equal). PEG polymers used for covalent attachment to a target molecule such as IL-15, and as described herein, generally possess low polydispersity values of preferably less than about 1.2, more preferably less than about 1.15, still more preferably less than about 1.10, for example, less than about 1.05, or less than about 1.03.

A “physiologically cleavable” or “hydrolyzable” or “degradable” bond is a relatively labile bond that typically reacts with water (i.e., is hydrolyzed) under physiological conditions and under any suitable method of hydrolysis. The tendency of a bond to hydrolyze in water may depend not only on the general type of linkage connecting two atoms within a given molecule but also on the substituents attached to these atoms and the overall structure of the molecule. Hydrolytically unstable or weak linkages may typically include but are not limited to carboxylate ester, phosphate ester, anhydrides, acetals, ketals, acyloxyalkyl ether, imines, orthoesters, peptides, oligonucleotides, thioesters, and carbonates.

An “enzymatically degradable linkage” means a linkage that is subject to degradation by one or more enzymes.

A “stable” linkage or bond refers to a chemical bond that is substantially stable in water, that is to say, does not undergo hydrolysis under physiological conditions to any appreciable extent over an extended period of time. Examples of hydrolytically stable linkages generally include but are not limited to the following: carbon-carbon bonds (e.g., in aliphatic chains), ethers, amides, amines, and the like. Generally, a stable linkage is one that exhibits a rate of hydrolysis of less than about 1-2% per day under physiological conditions. Hydrolysis rates of representative chemical bonds can be found in most standard chemistry textbooks.

The term “IL-15 moiety,” as used herein, refers to a peptide or protein moiety having human IL-15 activity. In addition, the term “IL-15 moiety” encompasses both the IL-15 moiety prior to conjugation to a PEG moiety as well as the IL-15 moiety following conjugation (i.e., covalent attachment) to (e.g., reaction with) a reactive PEG moiety such as, for example, mPEG-succinimidyl butanoate. As will be explained in further detail below, one of ordinary skill in the art can determine whether a given moiety has IL-15 activity. Proteins comprising an amino acid sequence corresponding to any one of SEQ ID NOs:1 through 3 are exemplary IL-15 moieties, as is any protein or polypeptide substantially homologous thereto. As used herein, the term “IL-15 moiety” includes such peptides and proteins modified deliberately, as for example, by site directed mutagenesis or accidentally through mutations. Included herewith are IL-15 sequences having from 1 to 6 additional glycosylation sites, sequences having at least one additional amino acid at the carboxy terminal end of the peptide or protein wherein the additional amino acid(s) includes at least one glycosylation site, and sequences having an amino acid sequence which includes at least one glycosylation site. The term is meant to include naturally, recombinantly and synthetically produced IL-15 moieties. Reference to a long-acting IL-15 receptor agonist is intended to encompass pharmaceutically acceptable salt forms thereof.

The term “substantially homologous” or “substantially identical” means that a particular subject sequence, for example, a mutant sequence, varies from a reference sequence by one or more substitutions, deletions, or additions, the net effect of which does not result in an adverse functional dissimilarity between the reference and subject sequences. For purposes herein, a sequence having greater than 95 percent homology (identity) under stringent conditions, equivalent biological activity (although not necessarily equivalent strength of biological activity), and equivalent expression characteristics to a given sequence is considered to be substantially homologous (identical). For purposes of determining homology, truncation of the mature sequence should be disregarded. Exemplary IL-15 polypeptides for use herein include those sequences that are substantially homologous to SEQ ID NO: 1. SEQ ID NO:2 is nearly identical to SEQ ID NO:1, with the exception that SEQ ID NO:2 has a methionine at the beginning of the sequence that is required for initiating translation in E. coli.

The term “fragment” means any protein or polypeptide having the amino acid sequence of a portion or fragment of the protein or polypeptide, e.g. an IL-15 moiety, and having the biological activity, or substantially the biological activity, of the protein or polypeptide, e.g. IL-15. Fragments include proteins or polypeptides produced by proteolytic degradation as well as proteins or polypeptides produced by chemical synthesis by methods routine in the art.

As used herein, the term “treating cancer” is not intended to be an absolute term, and may include, for example, reducing the size of a tumor or number of cancer cells, causing a cancer to go into remission, or preventing growth in size or number of cancer cells, and the like. In some circumstances, treatment in accordance with the instant disclosure leads to an improved prognosis.

As used herein in a method for treating a subject having cancer, the phrase “a subject in need of treatment” refers to an individual or subject that has been diagnosed with cancer.

As used herein, the term “enhancing”, for example, in the context of an enhanced response, refers to a subject's or tumor cell's improved ability to respond to treatment, e.g., as disclosed herein, when compared to a given baseline or reference therapy. For example, an enhanced response may comprise an increase in responsiveness of at least 5%, 10%, 15%, 20%, 25%, 30%, 35%, 40%, 45%, 50%, 55%, 60%, 65%, 70%, 75%, 80%, 85%, 90%, 95% or 98% or more, based upon any one or more indicators of responsiveness to treatment. As used herein, “enhancing” can also refer to enhancing the number of subjects who favorably respond to treatment, e.g., when compared to a given basis for such comparison.

“Refractory” as used herein refers to a disease, such as, for example, a cancer, that does not respond to a treatment. A refractory cancer can be resistant to a treatment before or at the beginning of the treatment, or, a refractory cancer can become resistant during a treatment. A refractory cancer is also called a resistant cancer.

“Relapsed” or a “relapse” as used herein refers to the reappearance of a disease (e.g., cancer) or the signs and symptoms of a disease such as cancer after a period of improvement or responsiveness, e.g., after prior treatment of a therapy, e.g., cancer therapy.

As used herein, the term “CD19” refers to the cluster of differentiation 19 protein, which is an antigenic determinant detectable on leukemia precursor cells. The human and murine amino acid and nucleic acid sequences can be found in a public database, such as, for example, GenBank, UniProt and Swiss-Prot. As used herein, “CD19” includes proteins comprising mutations, e.g., point mutations, fragments, insertions, deletions and splice variants of full length wild-type CD19. CD19 is expressed on most B lineage cancers, including, e.g., acute lymphoblastic leukemia, chronic lymphocyte leukemia and non-Hodgkin lymphoma.

The phrases “therapeutically effective”, “therapeutically effective amount”, “effective amount” or “in an amount effective” refer to a sufficient amount or dosage to promote a desired physiological response, such as, in the instance of administration of the long-acting IL-15 receptor agonist, an amount that is sufficient to promote an enhanced response to administration of the adoptive cellular immunotherapy composition comprising, e.g., CAR T cells. The precise amount will depend upon numerous factors, such as for example, the particular condition being treated, the patient population, individual patient considerations, the components and physical characteristics of the therapeutic composition and particular combination to be administered, the particular adoptive cell transfer therapy that is being administered (e.g., the particular composition of cells comprised in a CAR T cell composition, and/or the chimeric antigen receptor(s) that is/are expressed by the CAR T cells), and the like, and may be determined by one appropriately skilled in the art.

“Substantially” or “essentially” means nearly totally or completely, for instance, 95% or greater of a given quantity.

Similarly, “about” or “approximately” as used herein means within plus or minus 5% of a given quantity.

“Optional” or “optionally” means that the subsequently described circumstance may but need not necessarily occur, so that the description includes instances where the circumstance occurs and instances where it does not.

“Pharmaceutically acceptable excipient” or “pharmaceutically acceptable carrier” refers to a component that may be included in the compositions described herein and causes no significant adverse toxicological effects to a subject.

The term “patient,” or “subject” as used herein refers to a living organism suffering from or prone to a condition that can be prevented or treated by administration of a compound or composition or combination as provided herein, such as a cancer, and includes both humans and animals. Subjects include, but are not limited to, mammals (e.g., murines, simians, equines, bovines, porcines, canines, felines, and the like), and preferably are human (including pediatric and adult subjects).

### Overview

While a promising therapy for inducing an effective anti-tumor response in, e.g., hematological malignancies, following adoptive cell therapy with CAR T cells, patients frequently relapse after an initial favorable response to treatment. In an effort to address at least some of the shortcomings associated with current CAR T cell strategies, such as for example, by improving persistence, improving durability of response, overcoming or addressing resistance, improving safety, and/or improving patient outcome (efficacy), provided herein is a method comprising administering to a subject having cancer, an adoptive cellular immunotherapy composition comprising T cells that have been modified to express a chimeric antigen receptor, such as CD19-directed CAR T cells, and a long-acting IL-15 receptor agonist having features as described herein. In light of the disadvantages associated with current CAR T cell immunotherapies, further enhancements are needed to provide durable and effective responses to treatment. Thus, the present disclosure is based, at least in part, on the discovery of a particularly beneficial therapeutic immuno-oncology combination comprising CAR T cell therapy and administration of a long-acting IL-15 agonist, and more specifically, one that preferably retains receptor binding to IL-15 receptor a, as illustrated in an exemplary in vivo model, as will become apparent from the instant disclosure and supporting examples.

### Adoptive Chimeric Antigen Receptor Cell Transfer Therapy and Compositions

The treatment methods provided herein comprise administering ex vivo expanded, genetically engineered T cells transduced to express an artificial target tumor-associated antigen (TAA) binding domain, i.e., for stimulating a cancer specific-immune response. The compositions and methods provided herein find use in, among other things, both clinical and research applications. Without being bound by theory, it is believed that enhanced anti-tumor outcomes can be achieved via the IL-15 pathway (i.e., via administration, along with adoptive cell transfer, of a long acting IL-15 receptor agonist) to simulate the desired T cell response due to the complementary mechanisms of immune activation of adoptive cell transfer, e.g., CAR T cell transfer, and a long acting IL-15 receptor agonist as provided herein.

Any suitable chimeric antigen receptor T cell (CAR T cell) therapy can be used in the methods provided herein, and the disclosure is not limited in this regard. See for example, Rosenberg, S., et al., Adoptive Cell Transfer: A clinical path to effective cancer immunotherapy. Nat Rev Cancer. 2008 April; 8(4): 299-308 and Sadelain, M., et al, Current Opinion in Immunology, Vol 21 (2), 215-223 (2009); see also Kalos, M., et al., Sci Transl Med 2011; 3: 95ra73; and Grupp S A, et al, N Engl J Med 2013; 368:1509-1518. It will be appreciated that any suitable CAR T cell as known in the art may be used in the methods and therapies as described herein. Non-limiting examples of suitable CAR T cells and therapies for use herein include those described, for example, in U.S. Patent Publication Nos. 2017/0209492 and 2019/091308, and in U.S. Pat. Nos. 8,911,993; 8,975,071; 9,328,156; 9,987,308 and 10,253,086.

In one or more embodiments, the CAR T cells comprise an antigen binding domain that binds to a tumor antigen. In yet some further embodiments, the tumor antigen is selected from the group consisting of CD19, CD20, CD22, and ROR1, and combinations of the foregoing. In yet some further embodiments, the CAR T cells are CD19-targeted T cells comprising an antigen binding domain that binds to CD19. See, for example, Turtle, C. J., et al., Clinical Pharmacology & Therapeutics, 12 May 2016 (online). In addition to the exemplary publications provided the preceding paragraph, further illustrative CD19 CAR T cells, for example, CD19 CAR T cells of defined CD4+:CD8+ composition, are described, for example, in Turtle, C. J., J. Clin Invest. 2016; 126(6): 2133-2138. Further CAR T cells suitable for use in the methods and therapies described herein include, for example, the CD19-directed tisagenlecleucel (KYMRIAH®) and the CD19-directed axicabtagene ciloleucel (YESCARTA®), both of which are approved by the U.S. FDA for use in treating B cell malignancies. In yet some other embodiments, the CAR T cells express receptor tyrosine kinase-like orphan receptor 1 (ROR1), a tumor associated molecule expressed, for example, in prevalent B-lymphoid and epithelial cancers, as well in a subset of non-small cell lung cancer and triple negative breast cancer, but not in normal B cells. ROR1-specific CAR T cells useful in the methods and therapies described herein are described, for example, in Hudecek, M., Clinical Cancer Research, June 2013, 19(12), 3153-3164; Hudecek, M., et al., Blood 2010, 116:4532-4541; and Sprecht, J. M., et al., Cancer Research, 78 (13 Supplement):CT131, July 2018. In some embodiments, the cellular construct targets the Ig/Fz portion of the extracellular domain of ROR1 and contains 4-1BB/CD3ζ intracellular signaling domain. In some embodiments, the ROR1 CAR T manufacturing process utilizes autologous peripheral blood lymphocytes, separated into CD4 and CD8 subsets, which are independently cultured with anti-CD3/anti-CD28 beads and IL-2, then transduced with a lentiviral vector encoding the ROR1 CAR. In yet some further embodiments, The CAR T cell product is formulated in a 1:1 ratio of CD4′ and CD8+CAR T cells.

CAR T cell therapy generally includes administration of CAR T cells to treat a patient suffering from a cancer, particularly a cancer whose tumor cells express the subject tumor antigen. In some embodiments, the CAR T cells are prepared by a method as described herein or as known in the art. For instance, following isolation, host T cells are transduced to express a target tumor-associated antigen recognition domain, expanded, and re-infused into the subject. Prior to infusion, patient pre-conditioning may also be carried out, for example, using lymphodepleting nonmyeloablative chemotherapy (NMC) to suppress endogenous regulatory T cells and to provide an optimized environment for the infused CAR T cells; alternatively, cyclophosphamide or any other suitable conditioning agent may be used. Such pre-conditioning is useful for eliminating or substantially reducing numbers of Tregs (regulatory T cells) and lymphocytes, which compete with the transferred cells for homeostatic cytokines. The host cells may be isolated from a variety of sources, such as lymph nodes, e.g. inguinal, mesenteric, superficial distal auxiliary, etc.; bone marrow; spleen; or peripheral blood, as well as from the tumor, e.g. tumor infiltrating lymphocytes. The cells may be allogeneic or, preferably, autologous. For ex vivo stimulation, the host cells are aseptically removed, and are suspended in any suitable media, as known in the art. After transduction, the cells are stimulated and expanded using any of a variety of protocols, particularly using combinations of anti-CD3, B7, anti-CD28, etc. Suitable protocols for ex vivo expansion of host T cells are described in “Focus on Adoptive T Cell Transfer Trials in Melanoma”, Clinical and Developmental Immunology, Vol 2010, Art. ID 260267.

For example, adoptive cell transfer of CAR T cells may be carried out by (i) obtaining autologous lymphocytes from a mammalian subject such as a human, (ii) genetically engineering the autologous lymphocytes to express a target tumor-associated antigen (TAA) recognition or binding domain, (iii) culturing the genetically engineered lymphocytes to produce expanded CAR T cells, and (iv) administering the expanded CAR T cells to the subject (i.e., patient). Autologous adoptive cell therapy may also be performed by (i) genetically engineering autologous lymphocytes to express a TAA binding domain, (ii) culturing the genetically engineered lymphocytes to produce expanded CAR T cells; (iii) administering nonmyeloablative lymphodepleting chemotherapy (NMC) to the subject; and (iv) after administering NMC, administering the expanded CAR T cells. Autologous cells can be derived from blood or be cloned using autologous antigen presenting cells and tumor-derived peptides.

The CAR T cells may be prepared by any means as described herein or as known in the art. Methods for producing a CAR and/or CAR T cells are described herein and are also described in U.S. Pat. Nos. 6,319,494; 6,410,319; 7,446,179; 7,446,191; 7,514,537; 7,741,465; and 9,987,308; in U.S. Published Application Nos. 2016/0185861, 2017/0137783 and 2019/0091308, and in PCT Published Application Nos. WO 2010/065818, WO 2010/025177, and WO 2007/059298, the methods of which are incorporated herein by reference. Further methods for producing CAR T cells are described by Berger C. et al., J. Clinical Investigation, 118:1 294-308 (2008) and Wang et al. (Molecular Therapy—Oncolytics (2016) 3, 16015), which are hereby incorporated by reference.

The CAR T cells are able to redirect antigen recognition based on the binding specificity of the CAR. The CAR may provide targeting to any TAA such that, when the CAR T cell is bound to its cognate antigen on the tumor cell surface, the tumor cell is affected so that the tumor burden in a patient is reduced, diminished or eliminated. The T cells may be engineered to express one or more chimeric antigen receptors (CAR) using any methods as described herein or known in the art. In one embodiment, isolated T cells are genetically engineered to express CAR constructs by transfecting the T cells with an expression vector encoding the CAR construct. Methods for transducing the T cell population expressing the selected CAR construct are known in the art and described in Sambrook et al., “Molecular Cloning: A Laboratory Manual”, 4^{th} Edition, Cold Spring Harbor Laboratory Press (2012), which is incorporated by reference herein.

Generally, the CAR includes an extracellular recognition or binding region/domain or exodomain (e.g., a single-chain fragment variable region (scFV) of an antibody) that binds a TAA on the tumor cells, a transmembrane domain and optional intracellular domains that may provide signals for T cell activation to attack the tumor cells.

Generally, the CARs as described herein are directed against a molecule (e.g. a protein) that is expressed on the cell surface of a cancer or tumor cell. A number of TAAs are known in the art with non-limiting examples including phosphoproteins, transmembrane proteins, glycoproteins, glycolipids, and growth factors. Assays for determining whether a given compound is suitable for use as a CAR recognition region to any of the antigens or targets as described herein can be determined through routine experimentation by one of ordinary skill in the art. Any CAR as described herein or as known in the art may be used in the methods and cells and adoptive cellular immunotherapy composition as used herein. Exemplary CARs include those described in U.S. Pat. Nos. 7,446,190; 7,741,465; 9,499,629; 9,987,308; and 10,253,086.

In some embodiments, the CAR recognition domain targets an antigen expressed on the cell surface of B-cells. In some embodiments, the CAR includes an anti-CD19 recognition or binding domain. Illustrative CD19-CAR constructs include the following: (i) CD19-directed chimeric antigen receptor (CTL019) lentiviral vector (SCFv from which CAR antigen-recognition moiety derived: FMC63; costimulatory domain: 4-1BB), Maude, S L., et al. N Engl J Med 2014; 371 (16): 1507-1517); (ii) CD19-directed chimeric antigen receptor incorporating an anti-CD-19 single variable fragment plus TCR zeta and CD28 signaling domains, γ-retroviral vector (SCFv from which CAR antigen-recognition moiety derived: FMC63; costimulatory domain: CD28), Lee, D W et al., Lancet 2015; 385 (9967)517-528; murine step cell virus-based splice-gag vector, (iii) MSGV-FMC63-28Z encoding anti-CD19 CAR as described in Kochenderfer J N, et al., J Immunother 2009; 32(7):689-702; (iv) CD-19 specific CD28/CD3ζ dual-signaling CAR, 19-28z (Brentjens, R J., et al., Sci Trans Med, 20 Mar. 2013:5 (177):177, in addition to those described in Park, J H, et al., Blood, 30 Jun. 2016, 127 (26), p. 3312-3320, Table 1).

The human CD19 antigen is a 95 kDa glycoprotein belonging to the immunoglobulin superfamily. CD19 is used as a biomarker for normal and neoplastic B cells as well as for follicular dendritic cells. CD19 is expressed from early stages of pre-B cell development through terminal differentiation, regulating B lymphocyte development and function. Expression of CD19 is highly conserved on most B cell tumors including B cell lymphomas such as non-Hodgkin lymphoma. CD19 is also expressed in most types of leukemia including B cell leukemias, acute lymphoblastic leukemia (ALL), chronic lymphocytic leukemia (CLL), and Waldenstrom's Macroglobulinemia (WM). The majority of B cell malignances (lymphomas and leukemias) express CD19 at normal to high levels. In some embodiments, the CAR includes an anti-CD19 binding portion. In yet further embodiments, the combination of a long-acting IL-15 receptor agonist and CD19 directed CAR T cells are used in treatment of a B cell malignancy including, without limitation, non-Hodgkin lymphoma, acute lymphoblastic leukemia (ALL), chronic lymphocytic leukemias (CLL), and Waldenstrom's Macroglobulinemia (WM). In some preferred embodiments, the CD19 CAR T cell therapy comprises CD-19-directed genetically modified autologous T cells.

A barrier to durability of response of CD19 CAR T cell therapy has been identified as the downregulation of target antigen CD19 from the tumor cell surface (Shah, et al., Frontiers in Oncology (2019) Vol. 9, Article 146). In some embodiments, the CAR T cell therapy is a multi-targeted CAR T cell therapy that includes targeting to CD19 and one or more additional TAAs. In some embodiments, the CAR T cell therapy comprises at least a portion of a cell population expressing a CD19 recognition or binding domain as well as one or more additional TAAs. In some embodiments, the CAR T cell therapy includes targeting to a TAA selected from CD20, CD22, CD38, CD123, CD70, or CD30. In some embodiments, the CAR T cell therapy comprises two or more cell populations each population expressing different CARs. The cell populations may be administered as a mixture or may be co-administered sequentially. Preferably, the multi-targeted CAR T cell therapy comprises administration of anti-CD19 CAR T cells and CAR T cells directed to one or more additional TAAs selected from CD20, CD22, CD38, CD123, CD70, or CD30. In some other embodiments, the CAR T cell therapy includes targeting to a TAA that is ROR1.

In some embodiments, the CAR includes one or more intracellular co-stimulatory signaling domains. Exemplary co-stimulatory domains include, but are not limited to, CD28, CD123 or 4-1BB with CD3ζ.

Expansion of lymphocytes such as T cells can be accomplished by any of a number of methods as are known in the art. For example, T cells can be expanded using non-specific T cell receptor stimulation in the presence of feeder lymphocytes and interleukin-2 (IL-2), IL-7, IL-15, IL-21, or combinations thereof. The non-specific T cell receptor stimulus can include, for example, a stimulating amount of a mouse monoclonal anti-CD3 antibody (available, e.g., from LS Bio, Seattle Wash.). Alternatively, T cells can be rapidly expanded by stimulation of peripheral blood mononuclear cells (PBMC) in vitro with one or more antigens (including antigenic portions thereof, such as epitopes), which can be optionally expressed from a vector, such as an human leukocyte antigen A2 (HLA-A2) binding peptide, in the presence of a T cell growth factor, such as interleukin-2 or interleukin-15, with interleukin-2 being preferred. The in vitro-induced T cells are rapidly expanded by re-stimulation with the same antigen(s) of the cancer pulsed onto HLA-A2-expressing antigen-presenting cells. Alternatively, the T cells can be re-stimulated with irradiated autologous lymphocytes or with irradiated HLA-A2+ allogeneic lymphocytes and interleukin-2.

Specific tumor reactivity of the expanded T cells can be tested by any method known in the art, e.g., by measuring cytokine release (e.g., interferon-gamma) following co-culture with tumor cells. For example, the adoptive cell transfer may include enriching cultured T cells for CD8+ T cells prior to rapid expansion of the cells. Following culture of the T cells in a medium containing interleukin-2, the T cells are depleted of CD4+ cells and enriched for CD8+ cells using, for example, a CD8 microbead separation. In some embodiments, a T cell growth factor that promotes the growth and activation of the autologous T cells is administered to the subject either concomitantly with the autologous T cells or subsequently to the autologous T cells. The T cell growth factor can be any suitable growth factor that promotes the growth and activation of the autologous T cells. Examples of suitable T cell growth factors include interleukin (IL)-2, IL-7, IL-15, IL-12 and IL-21, which can be used alone or in various combinations, such as IL-2 and IL-7, IL-2 and IL-15, IL-7 and IL-15, IL-2, IL-7 and IL-15, IL-12 and IL-7, IL-12 and IL-15, or IL-12 and IL2.

Expression of the chimeric antigen receptor in the CAR T cells may be compared in untransduced (UnTd) and transduced (Td) cells by fluorescence-activated cell sorting analysis and by recognition in vitro of HLA-A2^{+ 526} melanoma line and not the HLA-A2⁻ 888 melanoma line (Rosenberg, S., et al., Nat Rev. Cancer, 2008 April; 84(4):299-308). Universal type T cells can also be used such as described by Qasim, W., et al., Sci. Transl. Med. 9, eaaj2013 (2017). For example, universal CAR19 T cells can be generated using TALEN-mediated cell engineering in combination with lentiviral transduction and used in adopted cell therapy. The cells are generated by lentiviral transduction of non-human leukocyte antigen-matched donor cells and simultaneous transcription activator-like effector nuclease (TALEN)-mediated gene editing of T cell receptor a chain and CD52 gene loci.

Prior to administering the CAR T cells, patients may be preconditioned with chemotherapy (e.g. cyclophosphamide and fludarabine as described, for example, in U.S. Pat. No. 9,855,298) in order to improve efficacy of the therapy. Without being limited as to theory, preconditioning with chemotherapy may create space for proliferation of the CAR T cells by depletion of the normal lymphocytes, may eliminate cytokine sinks to increase the availability of homeostatic cytokines that promote CAR T cell proliferation, and/or may decrease the number of immunosuppressive cells such as regulatory T cells (Tregs) and myeloid-derived suppressor cells (Nair et al.). It will be appreciated that any suitable chemotherapy methods may be used as known in the art.

The expanded cells are then administered to the host by infusion, e.g., intravenous or intra-arteria infusion or other suitable form of delivery, which typically lasts from about 30 to about 60 minutes, although shorter or longer durations may be utilized. Other routes of administration include intraperitoneal, intrathecal, and intralymphatic. The expanded cells are provided in a suitable medium that may optionally include any of a variety of pharmaceutically acceptable additives, binders, fillers, carriers, preservatives, stabilizing agents, emulsifiers, buffers, and the like. Diluents and excipients include water, saline, and glucose. Representative media include but are not limited to, for example, Multiple Electrolytes Injection, Type 1, USP having a nominal pH range of about 5.5 to 8.0; tissue culture media containing human serum or fetal bovine serum; or a xeno-free and serum-free medium such as, for example, PRIME-XV T Cell Expansion XSFM (Irvine Scientific). Commercially available media include, e.g., RPMI 1640 (Thermo Fisher Scientific, Waltham, Mass.), AIM V cell culture medium (Thermo Fisher Scientific, Waltham Mass.), and X-VIVO 15 (Lonza, Basel, Switzerland).

### IL-15 Receptor Agonist

The methods described herein comprise, in one or more embodiments, the administration of a long acting, IL-15 receptor agonist. A compound is considered to be a long-acting IL-15 receptor agonist in accordance with the present disclosure as long as, following administration to a subject, the agonist exhibits IL-15 agonism in vivo for an amount of time that is longer than is the case for administration of the same interleukin-15 receptor agonist moiety in unmodified form. Conventional approaches, such as those involving radiolabeling a compound, administering the compound in vivo, and determining its clearance, can be used to assess whether a compound is a long-acting IL-15 receptor agonist (i.e., has a clearance that is longer than that of unmodified IL-15 administered in the same in vivo system). For example, the long-acting nature of an IL-15 receptor agonist may be determined using flow cytometry to measure STAT5 phosphorylation in lymphocytes at various time points after administration of the agonist in mice. As a reference, the signal is lost by around 24 hours with IL-15, but is sustained for a period greater than that for a long-acting IL-15 agonist as described herein.

The long-acting, IL-15 receptor agonist may be in the form of a pharmaceutically-acceptable salt, and references to the long-acting IL-15 receptor agonist are intended to encompass pharmaceutically acceptable salts thereof. Typically, such salts are formed by reaction with a pharmaceutically-acceptable acid or an acid equivalent. The term “pharmaceutically-acceptable salt” in this respect, will generally refer to the relatively non-toxic, inorganic and organic acid addition salts. These salts can be prepared in situ in the administration vehicle or the dosage form manufacturing process, or by separately reacting a long-acting interleukin-15 receptor agonist as described herein with a suitable organic or inorganic acid, and isolating the salt thus formed. Representative salts include the hydrobromide, hydrochloride, sulfate, bisulfate, phosphate, nitrate, acetate, valerate, oleate, palmitate, stearate, laurate, benzoate, lactate, phosphate, tosylate, citrate, maleate, fumarate, succinate, tartrate, napthylate, oxylate, mesylate, glucoheptonate, lactobionate, and laurylsulphonate salts and the like. (See, for example, Berge et al. (1977) “Pharmaceutical Salts”, J. Pharm. Sci. 66:1-19). Thus, salts as described may be derived from inorganic acids such as hydrochloride, hydrobromic, sulfuric, sulfamic, phosphoric, nitric, and the like; or prepared from organic acids such as acetic, propionic, succinic, glycolic, stearic, lactic, malic, tartaric, citric, ascorbic, palmitic, maleic, hydroxymaleic, phenylacetic, glutamic, benzoic, salicyclic, sulfanilic, 2-acetoxybenzoic, fumaric, toluenesulfonic, methanesulfonic, ethane disulfonic, oxalic, isothionic, and the like.

An exemplary long-acting IL-15 receptor agonist is encompassed by the structure:

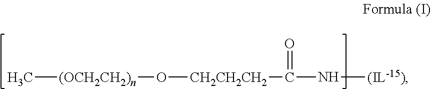

wherein IL-15 is an interleukin-15 moiety, (n) is an integer from about 150 to about 3,000, and ˜NH˜ represents an amino group of the IL-15 moiety. The above IL-15 receptor agonist is referred to herein as mono(methoxyPEG-N-butanamide)interleukin-15 (i.e., MPBA-IL15). An illustrative preparation of mono(methoxyPEG-N-butanamide)interleukin-15 is provided in Example 1.

In one or more embodiments related to the long-acting IL-15 receptor agonist of Formula (I), (n) is in a range from about 795 to about 1068. In some additional embodiments, (n) is in a range from about 840 to about 1023. In one or more particular embodiments, (n) has a value of, on average, about 907 or about 909, such that the average molecular weight of the polyethylene glycol chain is about 40,000 daltons.

Mono(methoxyPEG-N-butanamide)interleukin-15 (MPBA-IL15) is typically prepared as a distribution of predominantly monoPEGylated IL-15 (i.e., having a single methoxyPEG-N-butanamide moiety covalently attached to an amino group of IL-15, i.e., at a lysine or at the N-terminal alpha amine of IL-15), with minor amounts of di-PEGylated and higher PEGylated IL-15 species. Additional features of mono(methoxyPEG-N-butanamide)interleukin-15 are described, e.g., in International Patent Publication No. WO 2018/213341.

Illustrative compositions of MPBA-IL15 predominantly comprise the monoPEGylated species with less than about 10 mole % of the PEG dimer (i.e., having 2 methoxyPEG-N-butanamide moieties attached to IL-15), and even lower amounts of the higher PEGylated species (i.e. having 3 or more methoxyPEG-N-butanamide moieties attached to IL-15). Compositions of mono(methoxyPEG-N-butanamide)interleukin-15 will generally possess at least about 80 mole % monoPEGylated IL-15 species (based on all interleukin-15 species in the composition, including unmodified IL-15 (if present) and other IL-15 containing-species, e.g., diPEGylated IL-15 and higher). Compositions of MPBA-IL15 may preferably possess at least about 90 mole percent monoPEGylated IL-15 species, with less than about 10 mole % of other IL-15 containing species. In some embodiments, the MPBA-IL15 composition comprises less than about 5 mole % of PEG dimer (diPEGylated IL-15, having 2 methoxyPEG-N-butanamide moieties covalently attached to IL-15), and less than about 5 mole % of all other higher PEGylated species. In one or more embodiments, the MPBA-IL15 composition comprises at least about 85 mol %, 90 mol %, 95 mol %, 98 mol % or 99 mol % of the monoPEGylated species of Formula (I).

For example, in some preferred embodiments, the long-acting IL-15 receptor agonist composition comprises no more than about 20 mole percent (mol %) of long-acting IL-15 receptor agonists (of the IL-15 containing molecules in the composition), when considered collectively, encompassed by the formula:

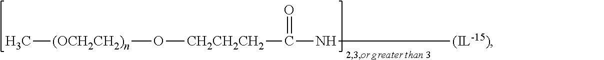

### Formula (II),

where the values of n are as previously described for all embodiments, or comprises no more than about 15 mole percent (mol %) of long-acting IL-15 receptor agonists (of the IL-15 containing molecules in the composition), when considered collectively, according to Formula (II), or contains no more than about 10 mole percent of long-acting IL-15 receptor agonists (of the IL-15 containing molecules in the composition), when considered collectively, according to Formula (II).

In some embodiments, the MPBA-IL15 composition comprises from about 0.1-15, 0.1-10, 0.1-5, 0.1-1, 1-20, 1-15, 1-10, 1-5, 5-20, 5-15, 5-10, 10-20, 10-15, or from about 15-20 mol % of compounds of Formula (II).

In some additional embodiments, the long-acting IL-15 receptor agonist composition comprises no more than about 1-5 mol % of free IL-15 protein (of the IL-15 containing molecules in the composition), when considered collectively.

In reference to the formulae above, “n” corresponds to an average number of (OCH₂CH₂) monomer subunits. MPBA-IL15 may be prepared, for example, using a suitably activated mPEG-butanoate ester reagent having an average molecular weight from about 6600 daltons to about 132,000 daltons. For example, MPBA-IL15 may be prepared using a suitably activated mPEG-butanoate ester reagent having an average molecular weight selected from e.g., 10 kD, 15 kD, 20 kD, 25 kD, 30 kD, 45 kD, 50 kD or 60 kD. The activated polymer reagent, when reacted with amino groups of IL-15 (e.g., lysines or the N-terminal), is effective to form a stable amide linkage between the IL-15 moiety and the polyethylene glycol moiety (moieties).

In one or more embodiments, n is an integer having a value that corresponds to a polyethylene glycol moiety having a weight average molecular weight selected from the group consisting of about 10,000 daltons (where n is ˜227), or about 15,000 daltons (where n is ˜340), or about 20,000 daltons (where n is ˜454), or about 25,000 daltons (where n is ˜568), or about 30,000 daltons (where n is ˜681), or about 40,000 daltons (where n is ˜909), or about 50,000 daltons (where n is ˜1136) or even about 60,000 daltons (where n is ˜1364) or greater.

An illustrative PEG reagent used to prepare MPBA-IL15 will typically possess a polydispersity value of about less than about 1.1, e.g., of about 1.05. Thus, in the case of a PEG reagent such as mPEG-succinimidyl butanoate having a nominal average weight of about 40,000 daltons, the PEG reagent (and resulting IL-15 conjugate) will possess covalently attached PEG moieties in a molecular weight range from about 35 kilodaltons to about 47 kilodaltons, or from about 37 kilodaltons to about 45 kilodaltons (41 kD±4 kDa). In some preferred embodiments, the mPEG butanoate activated ester reagent possesses a nominal average molecular weight of about 40 kilodaltons, i.e., where on average, n is ˜907-909.

When considering the IL-15 moiety, the term “IL-15 moiety” refers to the IL-15 moiety prior to conjugation as well as to the IL-15 moiety following conjugation. It will be understood, however, that when the original IL-15 moiety is attached to a polyethylene glycol moiety, the IL-15 moiety is slightly altered due to the presence of one or more covalent bonds associated with linkage to the polymer(s), such as in the case of the amide linkages formed during the preparation of MPBA-IL15.

The IL-15 moiety can be derived from non-recombinant methods and from recombinant methods, and the disclosure is not limited in this regard. In addition, the IL-15 moiety can be derived from human sources, animal sources (including insects), fungi sources (including yeasts), and plant sources.

The IL-15 moiety can be obtained according to the procedures described by, for example, Grabstein et al. (Grabstein et al. (1994) Science 264:965-968). The IL-15 moiety can also be prepared using recombinant methods, such as, for example, those described in EP Patent No. 0 772 624 B2 to Immunex Corporation. Alternatively, the IL-15 moiety can be purchased commercially from, for example, GenScript USA Inc. (Piscataway N.J.) and Peprotech (Rockyhill, N.J.).

More particularly, the IL-15 moiety can be expressed in bacterial [e.g., E. coli, see, for example, Fischer et al. (1995) Biotechnol. Appl. Biotechnol. 21(3):295-311], mammalian [see, for example, Kronman et al. (1992) Gene 121:295-304], yeast [e.g., Pichia pastoris, see, for example, Morel et al. (1997) Biochem. J. 328(1):121-129], and plant [see, for example, Mor et al. (2001) Biotechnol. Bioeng. 75(3):259-266] expression systems. The expression can occur via exogenous expression (when the host cell naturally contains the desired genetic coding) or via endogenous expression.

Further methods of preparation and/or purification of an IL-15 moiety are described in PCT Application No. PCT/US2018/032817.

Depending on the system used to express proteins having IL-15 activity, the IL-15 moiety can be unglycosylated or glycosylated and either may be used. That is, the IL-15 moiety can be unglycosylated or the IL-15 moiety can be glycosylated. In one or more embodiments, the IL-15 moiety is unglycosylated.

The IL-15 moiety can advantageously be modified to include and/or substitute one or more amino acid residues such as, for example, lysine, cysteine and/or arginine, in order to provide facile attachment of the polymer to an atom within the side chain of the amino acid. An example of substitution of an IL-15 moiety is described in U.S. Pat. No. 6,177,079. In addition, the IL-15 moiety can be modified to include a non-naturally occurring amino acid residue. Techniques for adding amino acid residues and non-naturally occurring amino acid residues are well known to those of ordinary skill in the art.

Exemplary IL-15 moieties are described herein, in the literature, and in, for example, U.S. Patent Application Publication No. US 2006/0104945, Pettit et al. (1997) J. Biol. Chem. 272(4):2312-2318, Wong et al., (2013) OncoImmunology 2(11), e26442:1-3, and in PCT Published Application No. WO 2018/213341. Preferred IL-15 moieties include those having an amino acid sequence comprising sequences selected from the group consisting of SEQ ID NOs: 1 through 3, and sequences substantially homologous thereto. A preferred IL-15 moiety has an amino acid sequence corresponding to SEQ ID NO: 1. In some embodiments, the IL-15 moiety is a functional homolog having at least about 85% or at least about 90% identity with any one of SEQ ID NOs: 1-3. In some embodiments, the IL-15 moiety is a functional homolog having at least about 95%, 98% or 99% identity with any one of SEQ ID NOs: 1-3.

In some instances, the IL-15 moiety will be in a “monomer” form, wherein a single expression of the corresponding peptide is organized into a discrete unit. In other instances, the IL-15 moiety will be in the form of a “dimer” (e.g., a dimer of recombinant IL-15) wherein two monomer forms of the protein are associated to each other.

In addition, precursor forms of IL-15 can be used as the IL-15 moiety. An exemplary precursor form of IL-15 has the sequence of SEQ ID NO: 3.

Truncated versions, hybrid variants, and peptide mimetics of any of the foregoing sequences can also serve as the IL-15 moiety. Biologically active fragments, deletion variants, substitution variants or addition variants of any of the foregoing that maintain at least some degree of IL-15 activity can also serve as an IL-15 moiety.

For any given peptide, protein moiety or conjugate, it is possible to determine whether that peptide, protein moiety or conjugate has a degree of IL-15 activity. Various methods for determining in vitro IL-15 activity are described in the art. An exemplary approach is based on a pSTAT assay. Briefly, if an IL-15-dependent CTLL-2 cell is exposed to a test article having IL-15 activity, initiation of a signaling cascade results that includes the phosphorylation of STAT5 at tyrosine residue 694 (Tyr694), which can be quantitatively measured. Assay protocols and kits are known and include, for example, the MSD Phospho(Tyr694)/Total STATa,b Whole Cell Lysate Kit (Meso Scal Diagnostics, LLC, Gaithersburg, Md.). For example, using this approach, a proposed IL-15 moiety that exhibits a pSTAT5 EC₅₀ value of no more than about 300 ng/mL (more preferably no more than about 150 ng/mL) at least one of 5 minutes or at 10 minutes is typically considered an “IL-15 moiety” in connection with the present disclosure. It is preferred, however, that the IL-15 moiety used is more potent (e.g., having a pSTAT5 EC₅₀ value of less than 150 ng/mL at one of least 5 minutes or 10 minutes, such as less than about 1 ng/mL, and even more preferably less than 0.5 ng/mL at least one of 5 minutes or at 10 minutes).

Other methodologies known in the art can also be used to assess IL-15 function, including electrometry, spectrophotometry, chromatography, and radiometric methodologies. See, for example, Ring et al. (2012) Nat. Immunol. 13(12):1187-1195 for one such additional type of assay.

As described previously, amino groups on the IL-15 moiety provide a site of attachment for reaction with the mPEG-succinimidyl butanoate reagent to provide an IL-15 receptor agonist encompassed by Formula (I). Considering the exemplary IL-15 amino acid sequences provided herein, it is evident that there are seven lysine residues each having an ε-amino acid that may be available for conjugation. Further, the N-terminal amine of methionine can also serve as a point of attachment to the PEG moiety. It will be appreciated that the polyethylene glycol moiety may be attached at any one or more of the lysine or the N-terminal amine positions. In some embodiments, a polyethylene glycol moiety attachment site is at one or more of Lys¹⁰ and Lys¹¹ (using the numbering as shown in SEQ ID NO: 2 as an example or Lys¹¹ and Lys¹² using SEQ ID NO:1). In some embodiments, a polyethylene glycol moiety is attached at the N-terminal amine. It will be appreciated that any of the lysine sites may be suitable as an attachment site (e.g. Lys³⁷ or Lys⁴² of SEQ ID NO:1) for the PEG moiety. In some embodiments, the MPBA-IL15 comprises a mixture of positional isomers, where covalent attachment of the polyethylene glycol moiety is predominately at the N-terminus (that is to say, of the collection of positional isomers, the isomer having the PEG moiety attached at the N-terminus is present in the highest amount, when compared to the other positional isomers).

MPBA-IL15 is immunotherapeutic agent that provides sustained IL-15 biological activity without the need for daily dosing through binding to all IL-15 receptor subunits (IL-15α, β and γ subunits). More particularly, MPBA-IL15, binds to the IL-15 receptor a and interleukin-2 (IL-2)/IL-15 βγ subunits and maintains the full spectrum of IL-15 biology, including pharmacodynamic (PD) effects on both NK cells and CD8+ memory T cells, whilst the polyethylene glycol moiety enlarges the hydrodynamic volume of the molecule, which serves to extend the effective half-life relative to unmodified rhIL-15.

In preclinical studies in rodents and non-human primates, MPBA-IL15 has been shown to have features considered by the inventors to be particularly advantageous when combined with CAR T cell therapy. For example, MPBA-IL-15 has been shown to (i) stimulate and expand NK cell proliferation, (ii) support CD8 T cell survival and memory formation without substantially inducing suppressive regulatory T cells, (iii) enhance formation of long-term immunological memory, and additionally, retains receptor binding to IL-R, but with an affinity that is less than that of unmodified IL-15.

Additional IL-15 receptor agonists that may also be suitable for use in the methods provided herein, include, for example, molecules such as N-803 (formerly ALT-803, a mutant IL-15/IL-15Rα fusion protein, see for example, Han, K., et al., Cytokine, 2011; 56(3):804-810; Zhu, X., et al., J Immunol. 2009; 183(6):3598-3607, and Xu, W., et al., Cancer Res. 2013; 73(10):3075-3086), NIZ985 (heterodimeric IL-15, IL-15/soluble IL-15Ra dimer, see, e.g., AACR; Cancer Res 2019; 79 (13 Suppl)), AM0015 (polyethylene glycol modified IL-15, see, e.g., WO 2017/112528), and OXS-3550 (single chain, tri-specific scFv recombinant fusion protein conjugate composed of the variable regions of the heavy and light chains of anti-CD16 and anti-CD33 antibodies and a modified form of IL-15, CAS Registry No. 2094086-30-1, UNI: E5GE91Q5FX).

### Methods

Based upon at least one or more of the features of the long-acting IL-15 receptor agonist, MPBA-IL15, in one aspect, provided herein are methods effective to induce an immune response in a cancer patient prior to administration of MPBA-IL15 by administering an adoptive cellular immunotherapy composition comprising autologous or allogenic, (preferably autologous) anti-tumor T cells that have been genetically transformed to express a chimeric antigen receptor that targets tumor cells, such as CD19 CAR T cells as previously described and having anti-tumor activity, accompanied/followed by administration of a long-acting IL-15 receptor agonist, i.e., MPBA-IL15, to thereby achieve an enhanced therapeutic effect. In preferred embodiments, the T-cell immunotherapy comprises CD19-directed genetically modified autologous T cells. Illustrative adoptive cellular immunotherapy compositions comprise tumor-reactive T cells modified to express a chimeric antigen receptor comprising an extracellular variable domain of an antibody directed to an antigen associated with the cancer (e.g., CD19), hinge and transmembrane domains, and an intracellular signaling domain of a T cell or other receptor, such as a costimulatory domain. For example, tumor-reactive T cells may be modified with a single chain antibody-derived chimeric antigen receptor directed to the CD19 molecule. In some embodiments, the cellular composition comprises CAR-modified cytotoxic tumor cells, e.g., CD19 CAR-modified CD8+ T lymphocytes, and may comprise, in addition, other types of T lymphocytes (e.g., helper T lymphocytes), e.g., CD4+ or other T cells that have been genetically modified to have a chimeric antigen receptor directed to the antigen associated with the cancer, e.g., CD19, and the disclosure is not limited in this regard (i.e., to the particular constitution of the adoptive cellular immunotherapy composition comprising CAR T cells, e.g., CD19-directed CAR T cells). Representative CAR T cell compositions suitable for use in the methods provided herein are described in the preceding sections.

In some embodiments, cells comprised in the adoptive cellular immunotherapy composition are formulated by first harvesting them from their culture medium, followed by washing and concentrating the cells in a medium and container system suitable for administration in a treatment-effective amount. A suitable infusion medium can be any isotonic medium formulation, such as for example, normal saline, RPMI 1640 (ThermoFisher), AIM V Serum Free Medium (ThermoFisher), and X-VIVO™ Media (Lonza Walkersville), 5% dextrose in water, or Ringer's lactate, among others.

The adoptive cellular immunotherapy composition comprising CAR T cells, e.g., CD19 CAR T cells, is typically administered in a treatment effective amount, that is, in an amount effective to confer immunity to the subject. By immunity is meant a lessening of one or more physical symptoms associated with a tumor or cancer to which the lymphocyte response is directed. The adoptive cellular immunotherapy composition is typically administered by infusion, with each infusion comprising from at least two cells to at least 10⁶ to 10¹⁰ cells/kg, preferably in a range of at least 10⁷ to about 10⁹ cells/kg, or preferably in a range of at least 10⁶ to about 10⁸ cells/kg. In some particular, but non-limiting embodiments, each infusion comprises at least about at least about 0.2×10⁶ cells/kg to about 6.0×10⁸ cells/kg, at least about 2.0×10⁶ cells/kg to about 2.0×10⁸ cells/kg, at least about 0.2×10⁶ cells/kg to about 5.0×10⁶ cells/kg, at least about 0.1×10⁸ cells/kg to about 2.5×10⁸ cells/kg, or at least about 0.6×10⁸ cells/kg to about 6.0×10⁸ cells/kg. The cells may be administered by a single infusion, or by multiple infusions. In some particular embodiments, the cells are administered as a single-dose infusion. Since different individuals vary in responsiveness, the number of cells infused, as well as the number of infusions and the time range over which multiple infusions are administered can be determined by a health care professional, as determined by routine examination. In some embodiments, administration of the T-cell immunotherapy is preceded by administration of a lymphodepleting chemotherapy regiment, such as for example, administration of cyclophosphamide (typically intravenously) and fludarabine (typically intravenously).

In accordance with the methods described herein, the long-acting IL-15 receptor agonist is administered in an amount effective to enhance the results of the CAR T cell immunotherapy. For confirmation, with respect to the long-acting IL-15 receptor agonist, MPBA-IL15, the amount and extent of the activation can vary widely and still be effective when coupled with administration of the adoptive cellular immunotherapy composition. That is to say, an amount of MPBA-IL15 that exhibits only minimal IL-15 receptor agonist activity for a sufficiently extended period of time can still be a long-acting IL-15 receptor agonist so long as when administered in combination with CAR T cell therapy, the methods described herein enable a clinically meaningful response. In some instances, due to (for example) synergistic interactions and responses, only minimal IL-15 receptor agonist activity may be required when accompanied by CAR T cell therapy, e.g., CD19 CAR T cell therapy, and it will be appreciated that the therapeutic amount of either or both of the long-acting IL-15 receptor agonist and the numbers of CAR T cells administered may be lower than the therapeutically effective amount of either component when administered alone.

As described herein, one aspect of the present disclosure provides a method that is useful for (among other things) treating a patient suffering from a condition, such as cancer, that is responsive to treatment with either or both of the adoptive cellular immunotherapy composition and the long-acting IL-15 receptor agonist. For example, patients may be responsive to the individual agents alone as well as the combination, but are more responsive to the combination. By way of further example, patients may be non-responsive to one of the individual immunotherapeutic agents, but are responsive to the combination. By way of still further example, patients may be non-responsive to either of the individual agents alone, but are responsive to the combination.

The long-acting IL-15 receptor agonist, MPBA-IL15, may be administered by any suitable means as known in the art. In some embodiments, the long-acting IL-15 receptor agonist is administered parenterally. As used herein, the term “parenteral” includes subcutaneous, intravenous, intra-arterial, intratumoral, intralymphatic, intraperitoneal, intracardiac, intrathecal, and intramuscular injection, as well as by infusion.

MPBA-IL15 may be combined with one or more suitable excipients or diluents to form a composition suitable for administration or further use. The long acting IL-15 receptor agonist may be comprised in a single dose composition, optionally accompanied by one or more pharmaceutically acceptable excipients. Suitable pharmaceutically acceptable excipients include those described, for example, in the Handbook of Pharmaceutical Excipients, 7^{th} ed., Rowe, R. C., Ed., Pharmaceutical Press, 2012. One such exemplary formulation comprises MPGA-IL15 in a solution comprising phosphate buffer and trehalose at a pH of 6.8. For example, an exemplary formulation comprises MPBA-IL15 formulated in a solution of potassium phosphate buffer, trelahose, and polysorbate 20 at a pH of about 6.

Suitable formulation types for parenteral administration include ready-for-injection solutions, dry powders for combination with a solvent prior to use, suspensions ready for injection, dry insoluble compositions for combination with a vehicle prior to use, and emulsions and liquid concentrates for dilution prior to administration, among others. In some particular embodiments, the long-acting IL-15 receptor agonist is provided in a formulation suitable for intravenous administration, and is administered intravenously. In some other embodiments, the long-acting IL-15 receptor agonist is provided in a formulation suitable for subcutaneous administration, and is administered subcutaneously. In some additional embodiments, the long-acting IL-15 receptor agonist is administered intratumorally. Other modes of administration are also contemplated, such as pulmonary, nasal, buccal, rectal, sublingual and transdermal.

Generally, a therapeutically effective amount of the long-acting IL-15 receptor agonist will range from about 5 mcg (μg) to about 10 mg, based upon dose of protein (IL-15 equivalents) administered. A given dose can be periodically administered up until, for example, the clinician determines an appropriate endpoint (e.g., cure, regression, partial regression, and so forth) is achieved.

In some embodiments, the therapeutically effective dose of the long-acting IL-15 receptor agonist, MPBA-IL15, ranges from about 0.10-70 mcg/kg (micrograms/kilogram, μg/kg IL-15 equivalents). In other embodiments, the therapeutically effective dose ranges from about 0.10 mcg/kg to about 50 mcg/kg, or from about 0.30 to about 45 mcg/kg, or from about 0.25 mcg/kg to about 0.1 mg/kg, about 0.01 mg/kg to about 0.1 mg/kg per day or about 0.03 mg/kg to about 0.1 mg/kg per day. In other embodiments, the therapeutically effective dose ranges from about 1-10 mcg/kg, about 0.03 mg/kg to about 0.1 mg/kg. In some specific, but non-limiting embodiments, the therapeutically effective dose is about 0.25 mcg/kg, 0.3 mcg/kg, 0.5 mcg/kg, 1 mcg/kg, 2 mcg/kg, 3 mcg/kg, 5 mcg/kg, 6 mcg/kg, 7 mcg/kg, 10 mcg/kg, 15 mcg/kg, 20 mcg/kg, 25 mcg/kg, 0.01 mg/kg, 0.03 mg/kg, 0.05 mg/kg, or 0.1 mg/kg.

In yet some further embodiments, the therapeutically effective dose of the long-acting IL-15R agonist ranges from about 0.25-25 μg/kg. In other embodiments, the therapeutically effective dose ranges (e.g. per day) from about 0.25 μg/kg to about 0.1 mg/kg, about 1.0 μg/kg to about 20 μg/kg, about 1.0 μg/kg to about 15 μg/kg, about 1.0 μg/kg to about 10 μg/kg, about 1.0 μg/kg to about 5.0 μg/kg, about 1 μg/kg to about 1.5 μg/kg, about 1.5 μg/kg to about 20 μg/kg, about 1.5 μg/kg to about 15 μg/kg, about 1.5 μg/kg to about 10 μg/kg, about 1.5 μg/kg to about 5.0 μg/kg, about 5.0 μg/kg to about 20 μg/kg, about 5.0 μg/kg to about 15 μg/kg, about 5.0 μg/kg to about 10 μg/kg, about 10 about 1.5 μg/kg to about 20 μg/kg, about 10 μg/kg to about 20 μg/kg, about 10 μg/kg to about 15 μg/kg, about 15 μg/kg to about 20 μg/kg, about 0.01 mg/kg to about 0.1 mg/kg per day or about 0.03 mg/kg to about 0.1 mg/kg per day. In other embodiments, the therapeutically effective dose ranges from about 1-10 μcg/kg, about 0.03 mg/kg to about 0.1 mg/kg. In some specific, but not limiting embodiments, the therapeutically effective dose is about 0.25 μg/kg, 0.3 μg/kg, 0.5 μg/kg, 1 μg/kg, 1.5 μg/kg, 2 μg/kg, 3 μg/kg, 5 μg/kg, 6 μg/kg, 7 μg/kg, 10 μg/kg, 15 μg/kg, 20 μg/kg, 25 μg/kg, 0.01 mg/kg, 0.03 mg/kg, 0.05 mg/kg, or 0.1 mg/kg per day.

As in the case of administration of the adoptive cellular immunotherapy composition, the dose of the long-acting IL-15 receptor agonist will vary, for example, depending upon the age, weight, and general condition of the subject as well as the severity of the condition being treated, the particular adoptive cellular immunotherapy composition, and the judgment of the health care professional.

In one or more embodiments, the adoptive cell transfer is carried out prior to administration of the long-acting, IL-15 receptor agonist. For example, the adoptive cell transfer-based cell infusion of CAR T cells may occur immediately, up to 1 hour, up to 2 hours, up to 3 hours, up to 4 hours, up to 5 hours, up to 6 hours, up to 7 hours, up to 8 hours, up to 9 hours, up to 10 hours, up to 11 hours, up to 12 hours, up to 1 day, up to 2 days, up to 3 days, up to 4 days, up to 5 days, up to 6 days, up to 7 days, up to 8 days, up to 9, days, up to 10 days, up to 11 days, up to 12 days, up to 13 days, up to 14 days, up to 15 days, up to 16 days, up to 17 days, up to 18 days, up to 19 days, up to 20 days, up to 21 days, up to 22 days, up to 23 days, up to 24 days, up to 25 days, up to 26 days, up to 27 days, up to 28 days, up to 29 days, up to 1 month, up to 3 months, up to 6 months or any combination thereof prior to administration of MPBA-IL15.

Alternatively, in some embodiments, the adoptive cell transfer is carried out following administration of the long-acting, IL-15 receptor agonist. For example, the adoptive cell transfer-based cell infusion of CAR T cells may occur immediately, up to 1 hour, up to 2 hours, up to 3 hours, up to 4 hours, up to 5 hours, up to 6 hours, up to 7 hours, up to 8 hours, up to 9 hours, up to 10 hours, up to 11 hours, up to 12 hours, up to 1 day, up to 2 days, up to 3 days, up to 4 days, up to 5 days, up to 6 days, up to 7 days, up to 8 days, up to 9, days, up to 10 days, up to 11 days, up to 12 days, up to 13 days, up to 14 days, up to 15 days, up to 16 days, up to 17 days, up to 18 days, up to 19 days, up to 20 days, up to 21 days, up to 22 days, up to 23 days, up to 24 days, up to 25 days, up to 26 days, up to 27 days, up to 28 days, up to 29 days, up to 1 month, up to 3 months, up to 6 months or any combination thereof, subsequent to administration of MPBA-IL15.

As used herein in reference to treatment of a subject having cancer, the terms “treatment,” “treat,” and “treating” are meant to include the full spectrum of intervention for the cancer from which the subject is suffering, such as administration of the combination to alleviate, slow, stop, or reverse one or more symptoms of the cancer or to delay the progression of the cancer even if the cancer is not actually eliminated. Treatment can include, for example, a decrease in the severity of a symptom, the number of symptoms, or frequency of relapse, e.g., the inhibition of tumor growth, the arrest of tumor growth, or the regression of already existing tumors.

For example, an improvement in the cancer or a cancer-related disease may be characterized as a complete or partial response. “Complete response” refers to an absence of clinically detectable disease with normalization of any previously abnormal radiographic studies, bone marrow, and cerebrospinal fluid (CSF) or abnormal monoclonal protein measurements. “Partial response” refers to at least about a 10%, 20%, 30%, 40%, 50%, 60%, 70%, 80%, or 90% decrease in all measurable tumor burden (i.e., the number of malignant cells present in the subject, or the measured bulk of tumor masses or the quantity of abnormal monoclonal protein) in the absence of new lesions. The term “treatment” contemplates both a complete and a partial response.

With regard to the frequency and schedule of infusion of the adoptive cellular immunotherapy composition and administering of the long acting, IL-15 receptor agonist, one of ordinary skill in the art will be able to determine an appropriate frequency. For example, in a treatment cycle, a clinician may conduct adoptive cell transfer of CAR T cells in combination with administration of the long acting, IL-15 receptor agonist, either concurrently with the adoptive cell transfer, or preferably, following adoptive cell transfer. For example, in some treatment modalities, the long acting, IL-15 receptor agonist is administered within about 7 days of adoptive cell transfer of the CAR T cells (e.g., on any one of days 1, 2, 3, 4, 5, 6, or 7). In some instances, the long acting, IL-15 receptor agonist, i.e., MPBA-IL15, is administered within 4 days of adoptive cell transfer, e.g., on any one of days 1, 2, 3, or 4. Based upon the long acting nature of MPBA-IL15, the IL-15 receptor agonist is typically administered relatively infrequently (e.g., once every three weeks, once every two weeks, once every 8-10 days, once every week, etc.).

Exemplary lengths of time associated with the course of therapy include about one week; about two weeks; about three weeks; about four weeks; about five weeks; about six weeks; about seven weeks; about eight weeks; about nine weeks; about ten weeks; about eleven weeks; about twelve weeks; about thirteen weeks; about fourteen weeks; about fifteen weeks; about sixteen weeks; about seventeen weeks; about eighteen weeks; about nineteen weeks; about twenty weeks; about twenty-one weeks; about twenty-two weeks; about twenty-three weeks; about twenty four weeks; about seven months; about eight months; about nine months; about ten months; about eleven months; about twelve months; about thirteen months; about fourteen months; about fifteen months; about sixteen months; about seventeen months; about eighteen months; about nineteen months; about twenty months; about twenty one months; about twenty-two months; about twenty-three months; about twenty-four months; about thirty months; about three years; about four years and about five years. Typically, a single round of adoptive cell transfer, e.g., of CD19 CAR T cells, is provided to a patient, followed by one or more doses of the long acting, IL-15 receptor agonist, MPBA-IL15, however, in some instances, one or more additional cycles of adoptive cell transfer may occur.

The treatment methods described herein are typically continued for as long as the clinician overseeing the patient's care deems the treatment method to be effective, i.e., that the patient is responding to treatment. Non-limiting parameters that indicate the treatment method is effective may include one or more of the following: tumor shrinkage (in terms of weight and/or volume and/or visual appearance); a decrease in the number of individual tumor colonies; a decrease in the number of cancer cells; tumor elimination; progression-free survival; appropriate response by a suitable tumor marker (if applicable), increased number of NK (natural killer) cells, increased number of T cells, increased number of memory T cells, increased number of central memory T cells, reduced numbers of regulatory T cells such as CD4+ Tregs, CD25+ Tregs, and FoxP3+ Tregs.

As described previously, the adoptive cell composition (e.g., comprising CAR T cells) and the long acting, IL-15 receptor agonist can be administered separately. Alternatively, if provision of adoptive cell transfer and administration of the long acting, IL-15 receptor agonist is desired to be simultaneous, either as an initial dose or throughout the course of treatment or at various stages of the dosing regimen—and the CAR T cells and the long acting IL-15 receptor agonist are compatible together and in a given formulation—then the simultaneous administration can be achieved via infusion of single dosage form/formulation (e.g., intravenous administration of an intravenous formulation that contains both immunological components).

The presently described methods and compositions can be used to treat a patient suffering from any condition that can be remedied or prevented by the methods provided herein, such as cancer. For example, the methods are useful in the treatment of, for example, solid tumor, hematologic malignancy (liquid cancer), or melanoma, among other conditions. Exemplary conditions are cancers, such as, for example, melanoma, kidney, non-small cell lung breast cancer (e.g., triple negative breast cancer), bladder, head and neck cancer, fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, colon carcinoma, pancreatic cancer, brain cancer, breast cancer, ovarian cancer, prostate cancer, squamous cell cancer, basal cell cancer, adenocarcinoma, sweat gland cancer, sebaceous gland cancer, papillary cancer, papillary adenocarcinomas, cystadenocarcinoma, medullary cancer, bronchogenic cancer, renal cell cancer, hepatoma, bile duct cancer, choriocarcinoma, seminoma, embryonal cancer, Wilms' tumor, cervical cancer, Hodgkin lymphoma, non-Hodgkin lymphoma, testicular cancer, lung cancer, small cell lung cancer, brain cancer, bladder cancer, epithelial cancer, glioma, astrocytoma, medulloblastoma, craniopharyngioma, ependymoma, pinealoma, hemangioblastoma, acoustic neuroma, oligodendroglioma, meningioma, melanoma, multiple myeloma, neuroblastoma, retinoblastoma and leukemias.

In some particular embodiments, the cancer is a solid cancer.

In yet some further embodiments, the cancer is selected from, for example, breast cancer, ovarian cancer, colon cancer, prostate cancer, bone cancer, colorectal cancer, gastric cancer, lymphoma, malignant melanoma, liver cancer, small cell lung cancer, non-small cell lung cancer, pancreatic cancer, thyroid cancers, kidney cancer, cancer of the bile duct, brain cancer, cervical cancer, maxillary sinus cancer, bladder cancer, esophageal cancer, and adrenocortical cancer.

In yet one or more further embodiments, the cancer is selected from melanoma, kidney, non-small cell lung, breast, bladder, head and neck, and colon cancer.

In one or more particular embodiments, the breast cancer is triple negative breast cancer. Triple negative breast cancers are highly aggressive tumors that lack estrogen receptors, progesterone receptors, and ERBB2 (HER2) gene amplification.

In some other embodiments, the cancer is lymphoma or leukemia.

In some further embodiments, the cancer is a B-cell malignancy selected from, without limitation, non-Hodgkin lymphoma (NHL), acute lymphoblastic leukemia (ALL), chronic lymphocytic leukemias (CLL) and Waldenstrom's Macroglobulinemia (WM), with patient populations encompassing both pediatric and adult patients.

The present methods are useful for enhancing the therapeutic effectiveness of adoptive cell transfer of CAR T cells, e.g., CD19 CAR T cells, for example, by improving the subject's response, by administration of a long acting IL-15 receptor agonist, MPBA-IL15. An enhanced response may be evaluated at any suitable time point during treatment, after a single round of treatment, after 2-3 cycles of treatment, etc., and by any of a number of suitable methods, including, for example, shrinkage of a tumor (partial response), i.e., an evaluation of tumor size or volume, disappearance of a tumor, a reduction in the number of cancer cells, a reduction in disease progression (cancer has not progressed), and analysis of one or more tumor test markers if appropriate. The comparison may be conducted in a human patient, or in a suitable animal model such as a suitable murine model of cancer.

In yet some further embodiments, the methods, kits, composition and combination provided herein are effective to stimulate T cell and/or NK cell activity and/or proliferation in a subject. In some embodiments, the method is effective, for example, when evaluated in a mouse model of the corresponding cancer, for increasing the number of CD8+ T cells in the subject. In yet some other embodiments, the method is effective, for example, when evaluated in a cancer mouse model of the corresponding cancer, to increase the number of NK cells in the subject.

Blood samples may be collected from the subject both before and during treatment to characterize and monitor CAR-T cells and assess the effects of therapy on the number and activation of immune cell populations, including, but not limited to, NK cells, CD8+ T cells, and CD8+ memory cells. Characterization and persistence monitoring of the genetically modified CD19-directed CAR-T cells may be performed in peripheral blood by quantitative polymerase chain reaction (qPCR) before and during treatment and CAR-T cell phenotype may also be assessed by flow cytometry. Blood samples may also be collected before and during treatment to determine changes in cytokine levels and to profile changes in gene expression in response to therapy. In addition, whole blood samples or PBMCs may be collected and used for evaluation of other immune functions.

Fresh tumor bone marrow biopsies before, during, and after treatment may be collected where feasible for characterization of tumor cells and immune system activation. Assessments may include evaluation of changes in tumor-specific protein markers and immune cell populations in the tumor microenvironment. Characterization and monitoring of the genetically modified CD19 CAR-T cells in bone marrow may be performed by quantitative polymerase chain reaction (qPCR) before and after treatment with administration the long acting IL-15 receptor agonist, e.g., MPBA-IL15.

Tumor biopsy collection may also be conducted. Biopsies should preferably be performed on lesions that have not been exposed to prior radiation. Biopsies may be obtained from non-target lesions, unless there are no other lesions suitable for biopsy. Pre- and post-treatment tumor tissue biopsies should preferably be taken from the same lesion, if feasible. In addition, biopsies may be taken from a distant, non-injected lesion to serve as a control biopsy. Tumor tissue biopsies may be used to characterize infiltrating immune cell populations with immunohistochemistry (IHC) and/or flow cytometry using a panel of markers (including, but not limited to, CD3, CD4, CD8, and CD56).

In some embodiments, the combination immunotherapy described herein, in contrast to many ACT-based therapies, is effective to increase the extent and persistence (i.e., retention) of exogenously delivered CAR T cells at a tumor site or within the blood, to thereby provide increased antitumor efficacy.

All articles, books, patents, patent publications and other publications referenced herein are incorporated by reference in their entireties. In the event of an inconsistency between the teachings of this specification and the art incorporated by reference, the meaning of the teachings and definitions in this specification shall prevail (particularly with respect to terms used in the claims appended herein). For example, where the present application and a publication incorporated by reference defines the same term differently, the definition of the term shall be preserved within the teachings of the document from which the definition is located.

### EXAMPLES

It is to be understood that the foregoing description as well as the examples that follow are intended to illustrate and not limit the scope of the disclosure. Other aspects, advantages and modifications within the scope of the invention(s) will be apparent to those skilled in the art to which the disclosure pertains.

### Materials and Methods

CAR T Cells: human CD19 CAR T cells T cells expressing a CD19/4-1BB/CD3ζ CAR were generated from healthy donors as previously described. See, e.g., Turtle, C. J., et al. J. Clin Invest. 2016; 126(6): 2123-2138 and U.S. Pat. No. 9,987,308. Briefly, CD4 and CD8 T cells were isolated and separately transduced with CD19 CAR lentiviral vector (FIG. 2), transduced cells were sorted and then expanded with LCL (lymphoblastoid B-cell line) cells for 14-16 days; cells were assayed on day 15.

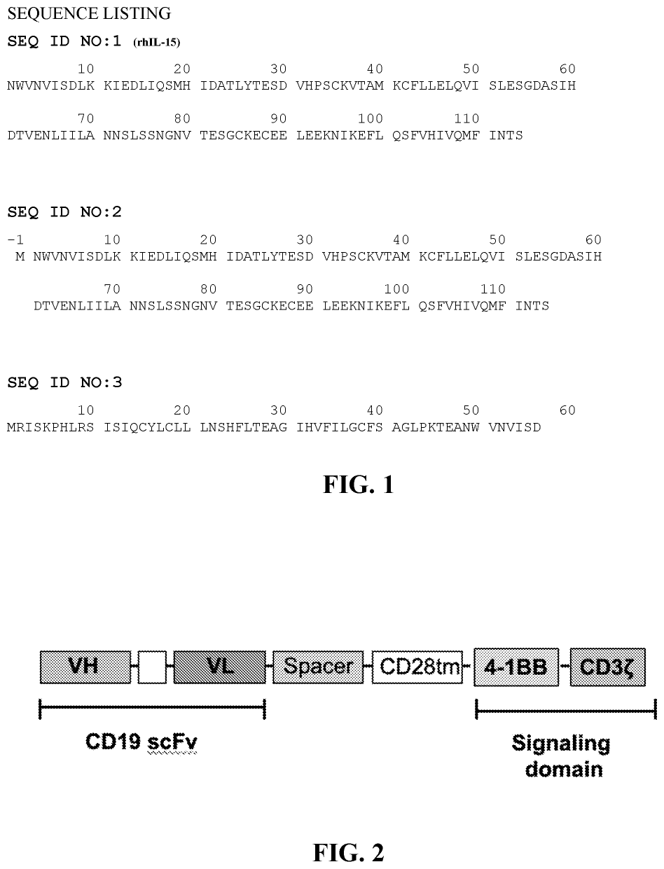

Recombinant IL-15 (“rIL-15”) SEQ ID NO:1 (as provided in FIG. 1), prepared using conventional techniques, was used in the following examples, although any suitable IL-15 moiety may be similarly employed. SEQ ID NO:1, a non-glycosylated recombinant human IL-15 expressed and purified from E. coli inclusion bodies, contains 115 amino acids and possesses two disulfide bridges, and has a molecular weight of about 12.9 kDa. The rIL-15 sequence includes an added N-terminal methionine not present on the native secreted human IL-15.

The reactive linear polymer reagent, mPEG-succinimidyl butanoate, 40 kDa (“mPEG-SBA”), CAS No. 187848-51-7, has the following structure,

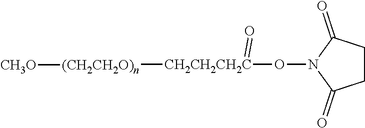

where n corresponds to an average number of monomer subunits to provide a polymer having a nominal average molecular weight of about 40 kilodaltons, i.e., where on average, n is ˜907-909. The PEG reagent possesses a polydispersity value of about less than about 1.1, e.g., of about 1.05, such that the nominal average molecular weight ranges from about 37 kilodaltons to about 45 kilodaltons (41 kD±4 kDa). The mPEGSBA is typically in the form of a white to off-which powder. Additional mPEG-succinimidyl butanoate reagents suitable for use include those having weight average molecular weights, for example, of about 10 kD, 15 kD, 20 kD, 25 kD, 30 kD, 45 kD, 50 kD or 60 kD. This activated polymer reagent, when reacted with amino groups of IL-15 (e.g., lysines or the N-terminal), is effective to form a stable amide linkage between the IL-15 moiety and the polyethylene glycol moiety.

Mono(methoxyPEG-N-butanamide)Interleukin-15 can be prepared, e.g., as described in Example 1 below. Mono(methoxyPEG-N-butanamide)Interleukin-15 (which may be referred to herein as MPBA-IL15) is a distribution of predominantly monoPEGylated IL-15, with a single mPEG-N-butanamide moiety covalently attached at a lysine or at the N-terminal alpha amine of IL-15, with minor amounts of di-PEGylated and higher PEGylated IL-15 species (CAS Registry No. 2361317-09-9). Additional features of mono(methoxyPEG-N-butanamide)interleukin-15 are described, e.g., in International Patent Publication No. WO 2018/213341 (the contents of which is incorporated herein by reference).

Potency Bioassay: The potency of MPBA-IL15 was determined by the phosphorylation of STAT5 in CTLL-2 cells, a murine T lymphocyte cell line which expresses IL-15α subunit, using the pSTAT5/total STAT5 multiplexed assay (Meso Scale Discovery, MD). Following receptor binding on CTLL-2 cells, downstream cell signaling activates STAT5 through phosphorylation to promote gene expression to induce cell proliferation. For the potency assay, phosphorylation of the receptor downstream STAT5 signaling molecule upon ligand binding was assessed to measure the biological response in short period of time.

Reference material, assay control, and test samples were serially diluted to a 10-fold final concentration using assay medium and then applied to a fixed number of cells for 10-minute incubation at 37° C./5% CO₂. The phospho- and total STAT5 were measured using the phospho-STAT5/total STAT5 multiplexed assay (Meso Scale Discovery, MD). A dose-dependent % phosphoprotein response curve was generated by non-linear regression analysis using a 4-parameter model. Parallel line analysis (PLA) software was used to assess the parallelism, significance of regression and to calculate the relative potency of the sample in relation to the reference material in the same plate.

Reverse phase HPLC was used to assess purity of MPBA-IL15 by reverse phase HPLC using a HALO Protein C4 analytical, a column temperature of 25° C., a flow rate of 1.0 mL/min and with a linear gradient of water/acetonitrile/trifluoroacetic acid (TFA), using ultraviolet (UV) detection at 214 nm.

Size exclusion HPLC was used to assess the relative purity of MPBA-IL15 using a Shodex Protein KW-803 column operating at 25° C. with a flow rate of 0.5 mL/min. Chromatographic elution was carried out using 15 mM Sodium Phosphate, pH 7.2 in acetonitrile, with UV detection at 214 nm.

Ion exchange HPLC was also used to assess the relative purity of MPBA-IL15 by separating charge variants that are acidic and basic using an Agilent PL-SAX column operating at 40° C. with a flow rate of 1.0 mL/minute. Elution was carried out using a linear gradient of bisTris Propane, pH 6.8: isopropyl alcohol and bisTris Propane, pH 6.8 with a solution of NaCl:isopropyl alcohol and UV detection at 214 nm.

### Example 1

### Preparation of a Long Acting IL-15 Receptor Agonist, Mono(Methoxypeg-N-Butanamide)_{40KD} interleukin-15

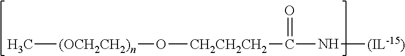

Preparation 1: A 2.7 ml solution of rIL-15 (1.23 mg/ml in PBS buffer, pH 7.4) was transferred to a small reaction vial. 300 μl of 0.6M borate buffer at pH 8 was added to adjust the pH to pH 8. mPEG-SBA, 40 kDa (nominal average molecular weight), stored at −20° C. under nitrogen, was warmed to ambient temperature. A ten-fold excess (relative to the molar amount of IL-15) of mPEG-SBA-40K was dissolved in 2 mM HCl to form a 10% PEG reagent solution. The 10% PEG reagent solution was quickly added to the IL-15 solution and mixed well. After the addition of the mPEG-SBA-40K, the pH of the reaction mixture was determined and adjusted to pH 8 using conventional techniques. To allow for coupling of the mPEG-SBA-40K to IL-15 (i.e., via formation of a stable amide linkage), the reaction solution was placed on a Slow Speed Lab Rotator for 1.5 hours to facilitate conjugation at room temperature. The reaction was quenched by addition of a solution of glycine.

The reaction yielded 40% mono-conjugate (i.e., having a single PEG moiety attached to IL-15), 24% di-conjugate (having two PEGs attached to IL-15) and 6% tri-conjugate (having three PEGs attached to IL-15) species. Approximately 30% unreacted IL-15 remained in the reaction mixture although reaction conditions were not optimized.

The mono-conjugate was separated/isolated by anion-exchange chromatography using a Q Sepharose High Performance column and sodium phosphate buffers as the eluent phase. The purified mono-mPEG-SBA40K-IL-15 conjugate (also referred to herein as mono-mPEG-butanamide-40K-IL-15 or mono(methoxyPEG-N-butanamide)_{40KD} interleukin-15, or mono-mPEG_{40K}-C4-amide-IL-15), was characterized by HPLC and SDS-PAGE. For the remainder of the Examples, purified mono-mPEG-SBA40K-IL-15 is referred to as Conjugate 1.

As indicated by the SDS gel, the purified conjugate was determined to possess a high level of purity and was absent detectable amounts of unreacted IL-15. Based upon the HPLC plot, the purified mono-mPEG-SBA-40K-IL-15 composition contained less than about 10% (molar amount) of di- or higher level conjugates.

Using this synthetic approach, conjugates such as mono-mPEG-SBA-10K-IL-15, mono-mPEG-SBA-15K-IL-15, mono-mPEG-SBA-20K-IL-15, mono-mPEG-SBA-25K-IL-15, mono-mPEG-SBA-30K-IL-15; mono-mPEG-SBA-45K-IL-15; mono-mPEG-SBA-50K-IL-15; and mono-mPEG-SBA-60K-IL-15 are prepared using mPEG-SBA having different nominal average molecular weights (e.g., having nominal average molecular weights of about 10 kilodaltons, 15 kilodaltons, 20 kilodaltons, 25 kilodaltons, 30 kilodaltons, 45 kilodaltons, 50 kilodaltons, 60 kilodaltons, and the like, respectively).

Preparation 2: An approximately 2 mg/ml solution of rIL-15 in a buffer (50 mM sodium phosphate, 100 mM sodium chloride, 10% sucrose, pH 7.4) was transferred to each of two different reaction vessels (referred herein as Composition 1 and Composition 2). To adjust the pH to 8.0, a Borate buffer (0.4M or 0.6M) at pH 8 was added. A ten-fold excess (relative to the molar amount of IL-15) of mPEG-SBA-40K (mPEG-SBA, 40 kDa), diluted in 2 mM HCl, was added to each of the IL-15 solutions and mixed well. After the addition of the mPEG-SBA-40K, the pH of the reaction mixture was determined to be pH 8, or adjusted if necessary by use of additional borate buffer. The final concentration of the rIL-15 in the reaction was targeted for 1 g/L, if necessary using an additional diluent (a buffer containing 50 mM sodium phosphate, 100 mM sodium chloride, 10% sucrose, pH 7.4 was used for Composition 1 and water was used for Composition 2). To allow for coupling of the mPEG-SBA-40K to IL-15 (i.e., via formation of predominantly stable amide linkages), the reaction solution was mixed for 45 or 60 minutes to facilitate conjugation at room temperature for Composition 1 or Composition 2, respectively. The reaction was quenched by addition of a 71-fold excess of glycine (relative to the molar amount of PEG initially added to the reaction at pH 8.0 for 30 minutes. For Composition 1, the pH was adjusted by titrating to pH 7.0 using a 0.2 M phosphoric acid.

The resulting composition was characterized by reverse phase HPLC (RP-HPLC), SDS-PAGE, and ion exchange HPLC (IEX-HPLC). The results of the RP-HPLC analysis are provided in Table IA below.

TABLE 1A

| | | Average % |
|---|---|---|
| | Component | Area |
| Composition 1 | IL-15 | 0.57 |
| | Mono-PEGylated | 82.66 |
| | IL-15 | |
| | RRT1 = Peak T1 | 8.05 |
| | (not characterized) | |
| | RRT2 = Peak T2 | 5.17 |
| | (not characterized) | |
| | RRT3 = Peak T3 | 3.57 |
| | (not characterized) | |
| Composition 2 | IL-15 | 0.36 |
| | Mono-PEGylated | 92.17 |
| | IL-15 | |
| | RRT1 = Peak T1 | 2.26 |
| | (not characterized) | |
| | RRT2 = Peak T2 | 3.28 |
| | (not characterized) | |
| | RRT3 = Peak T3 | 1.93 |
| | (not characterized) | |

RRT is relative retention time.

The results of the SEC-HPLC analysis are provided in Table 1B, below.

TABLE 1B

| | Component | Average % Area |
|---|---|---|
| Composition 1 | BMW species (tri-PEGylated | 5.56 |
| | IL-15 or greater “highmers”) | |
| | Di-mPEG IL-15 (di- | 15.46 |
| | PEGylated IL-15) | |
| | Mono-mPEG IL -15 | 78.98 |
| | (monomer) | |
| Composition 2 | HMW mPEG species | 5.25 |
| | di-mPEG IL-15 | 6.62 |
| | Mono-mPEG IL-15 | 88.14 |

HMW = high molecular weight mPEG-IL-15 conjugates having 3 or more PEGs covalently attached to interleukin-15.

The results of the IEX-HPLC analysis are provided in Table 1B, below.

TABLE IC

| | | Average % Area |
|---|---|---|
| Composition 1 | Basic region | 10.20 |
| | Main peak | 68.53 |
| | Acidic region | 21.29 |
| Composition 2 | Basic region | 10.43 |
| | Main peak | 56.32 |
| | Acidic region | 33.26 |

The compositions prepared predominantly comprised the mPEG-SBA-40K monoPEGylated species with less than about 10 mole % of the PEG dimer (i.e., having 2 PEG moieties attached to IL-15) and even lower amounts of the higher PEG species (i.e. having 3 or more PEG moieties attached to IL-15). Otherwise stated, compositions of mono(methoxyPEG-N-butanamide)interleukin-15 will generally possess at least about 80 mole % monoPEGylated IL-15 species (based on all interleukin-15 species in the resulting composition, including unmodified IL-15 and other IL-15 containing-species, e.g., diPEGylated IL-15 and higher), and may preferably possess at least about 90 mole % monoPEGylated IL-15 species, with less than about 10 mole % of other IL-15 containing species. In some embodiments, the mono(methoxyPEG-N-butanamide)interleukin-15 composition comprises less than about 5 mole % of PEG dimer (diPEGylated IL-15, having 2 methoxyPEG-N-butanamide moieties covalently attached to IL-15), and less than about 5 mole % of all other higher PEGylated species.

Two additional syntheses of mono(methoxyPEG-N-butanamide)interleukin-15 were prepared and analyzed. A summary of the analytical results is provided below in Table 1D.

MPBA-IL15 may be formulated for further use as a solution containing MPBA-IL15 at a concentration of 1 mg/mL (on a protein basis) in 10 mM potassium phosphate, 260 mM of trehalose and 0.02 w/v % polysorbate 20 at a pH of 6.8.

TABLE ID

| Method | Analyte | | Lot 1 | Lot 2 |
|---|---|---|---|---|
| RP-HPLC | Main Peak | ≥85% | 98 | 97 |
| | Di-PEG | Report Results (%) | 2 | 2.8 |
| | Free rhIL15 | Report Results (%) | <1 | 0.57 |
| SEC-HPLC | Main Peak | ≥90% | 98 | 100 |
| | Di-PEG | Report Results (%) | 2 | <3 |
| | HMW | Report Results (%) | ND | ND |
| IEX-HPLC | Main Peak | ≥60% | 78 | 79 |
| | Basic Region | Report Results (%) | 12 | 12.0 |
| | Acidic Region | Report Results (%) | 10 | 8.7 |

| | | | |
|---|---|---|---|
| Free PEG RP- | ≤5% wt/wt | 0.9% | 2% (1.57%) |
| HPLC/ELSD | | | |

Abbreviations:
NT = not tested;
Di-PEG = diPEGylated species;
HMW = higher molecular weight;
ND = not detected;
LOQ = limit of quantitation;
NTU = nephelometric turbidity units

### Example 2

### In Vitro Investigation of Mono(Methoxypeg-N-Butanamide)Interleukin-15 on Phosphorylation and Proliferation of CAR T Cells

The effects of mono(methoxyPEG-N-butanamide)interleukin-15 on human CD19 CAR T cells in vitro were explored as described below.

For in vitro studies, CAR T cells were incubated with mono(methoxyPEG-N-butanamide)interleukin-15 (0-100 ng/mL) with and without CD19 antigen. STAT5 phosphorylation and CFSE dilution were assessed by flow cytometry.

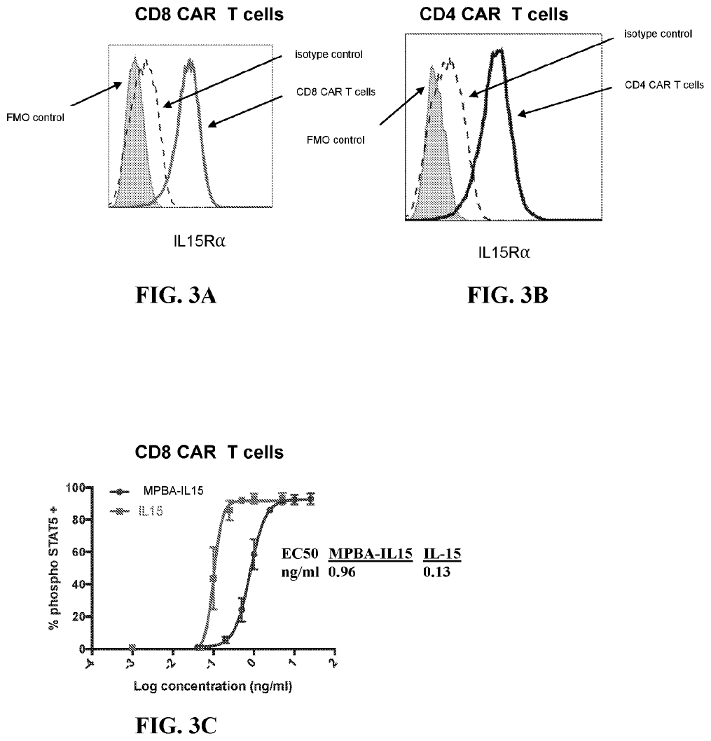

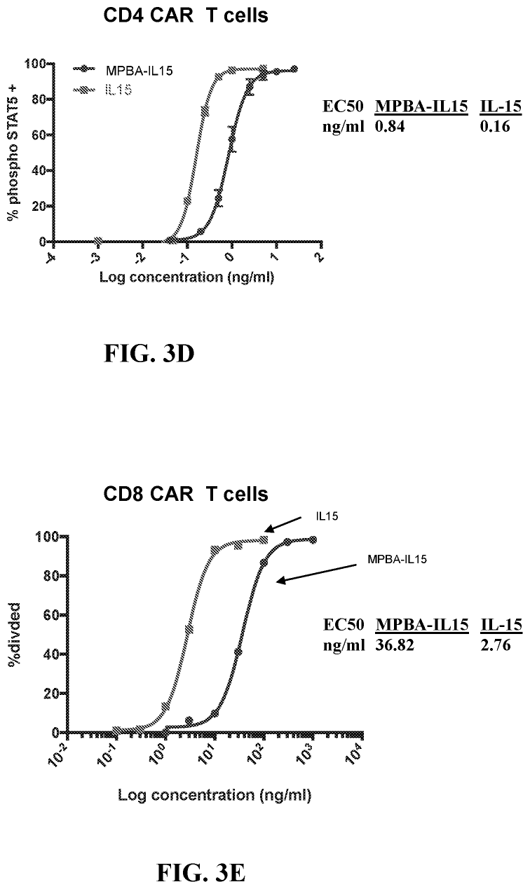

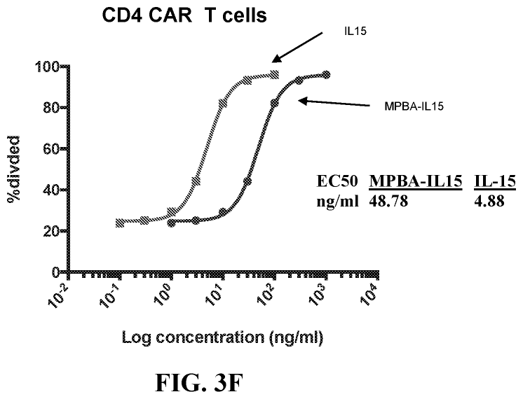

IL15Rα expression was measured by flow cytometry for CD8 CAR T cells as shown in FIG. 3A (CD8, solid green line, far right) and for CD4 CAR T cells as shown in FIG. 3B (CD4, solid blue line, far right). Also shown in each figure are FMO (gray-fill) and isotype (dashed line) controls. The CD8 and CD4 CAR T cells express IL-15Ra.

Dose dependent phosphorylation of STAT5 in response to mono(methoxyPEG-N-butanamide)interleukin-15 (MPBA-IL15) for both CD8 CAR T cells and for CD4 CAR T cells is shown in FIGS. 3C and 3D, respectively. CAR T cells were stimulated with various concentrations of MPBA-IL15 or IL-15 for 20 minutes. EC₅₀ values (ng/ml) for CD8 and CD4 CAR T cells in response to either IL-15 or mono(methoxyPEG-N-butanamide)interleukin-15 in a STAT-5 phosphorylation assay is summarized below:

TABLE 2

| STAT-5 | EC50, ng/ml | EC50, ng/ml |
|---|---|---|
| Phosphorylation | MPBA-IL15 | IL-15 |
| CD8 CAR T cells | 0.96 | 0.13 |
| CD4 CAR T cells | 0.84 | 0.16 |

Proliferation of CAR T cells labeled with CFSE and incubated with various concentrations of MPBA-IL15 or IL-15 for 4 days was also assayed by flow cytometry. Results are shown in FIGS. 3E (CD8 CAR T cells) and 3F (CD4 CAR T cells). EC50 values are summarized below.

TABLE 3

| | EC50, ng/ml | EC50, ng/ml |
|---|---|---|
| Proliferation | MPBA-IL15 | IL-15 |
| CD8 CAR T cells | 36.82 | 2.76 |
| CD4 CAR T cells | 48.78 | 4.88 |

For FIGS. 3C, D, E, and F, squares correspond to IL-15 and circles correspond to MPBA-IL15.

As described above, in vitro, mono(methoxyPEG-N-butanamide)interleukin-15 induces STAT5 phosphorylation and antigen-dependent proliferation of both CD8 and CD4 CD 19 CAR T cells in a dose-dependent manner.

### Example 3

### Investigation of Mono(Methoxypeg-N-Butanamide)Interleukin-15 on Efficacy of CD19 CAR T Cell Immunotherapy in a Preclinical Murine Lymphoma Model

Mono(methoxyPEG-N-butanamide)interleukin-15 (e.g., described in Example 1 above) retains binding affinity to IL-15Rα and exhibits reduced clearance, providing sustained pharmacodynamic responses. The effects of mono(methoxyPEG-N-butanamide)interleukin-15 on human CD19 CAR T cells in an in vivo xenogeneic B cell lymphoma model were explored in experiments as described below.

General Method: For in vivo studies, NSG mice received 5×10⁵ Raji lymphoma cells stably transduced to express firefly luciferase intravenously on day (D)-7, followed by infusion of a sub-therapeutic dose (0.8×10⁶) of CAR T cells (1:1 CD4:CD8) on DO alone or in combination with mono(methoxyPEG-N-butanamide)interleukin-15 (0.03, 0.10 or 0.30 mg/kg infused intravenously) administered weekly starting on D−1, 7, or 14. Tumor-free mice were rechallenged with Raji cells on D38. Tumors were assessed by bioluminescence imaging of the mice weekly. Results are illustrated in FIG. 10.

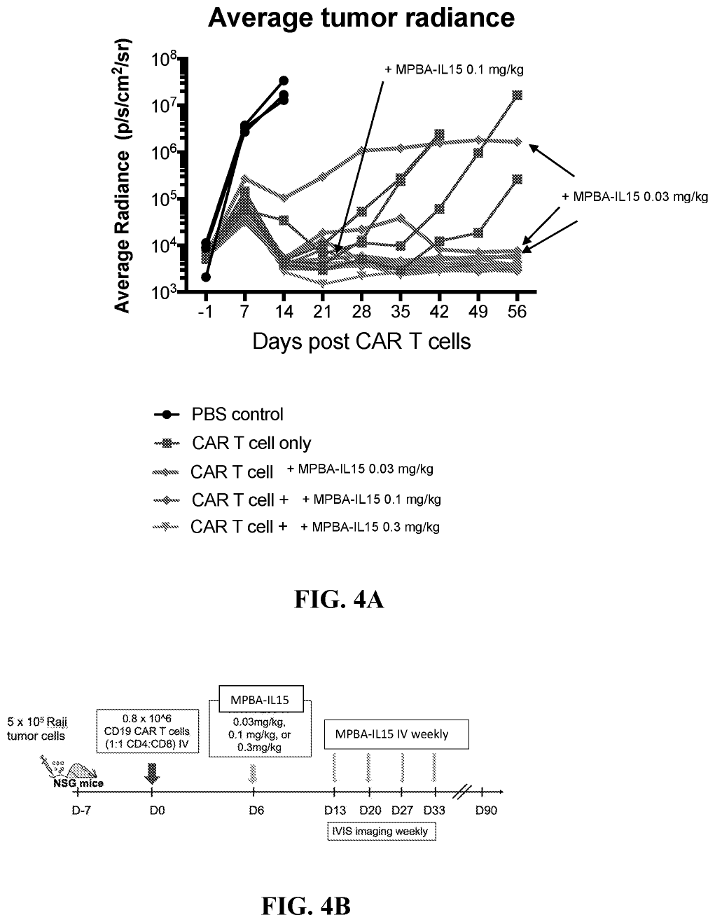

As shown in FIG. 4A (average tumor radiance versus days post CAR T cell administration for the various treatment groups), treatment with mono(methoxyPEG-N-butanamide)interleukin-15 at 0.10 mg/kg and 0.30 mg/kg in combination with CAR T cells leads to decreased tumor burden and eradication of Raji lymphoma in NSG mice when compared to CAR T cell therapy alone. In this study (Study A), Raji-bearing NSG mice received a sub-therapeutic dose of CAR T cells on DO followed by 0.030, 0.10 or 0.30 mg/kg of mono(methoxyPEG-N-butanamide)interleukin-15 starting on D6, and weekly thereafter (D13, D20, D27, D33, etc.). The treatment regimen is illustrated in FIG. 4B.

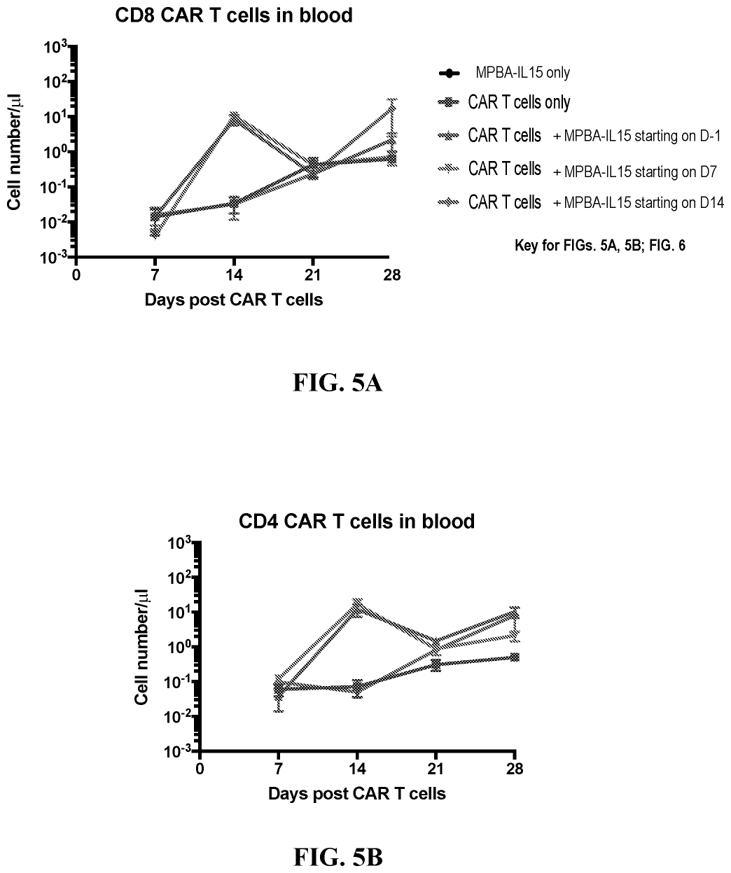

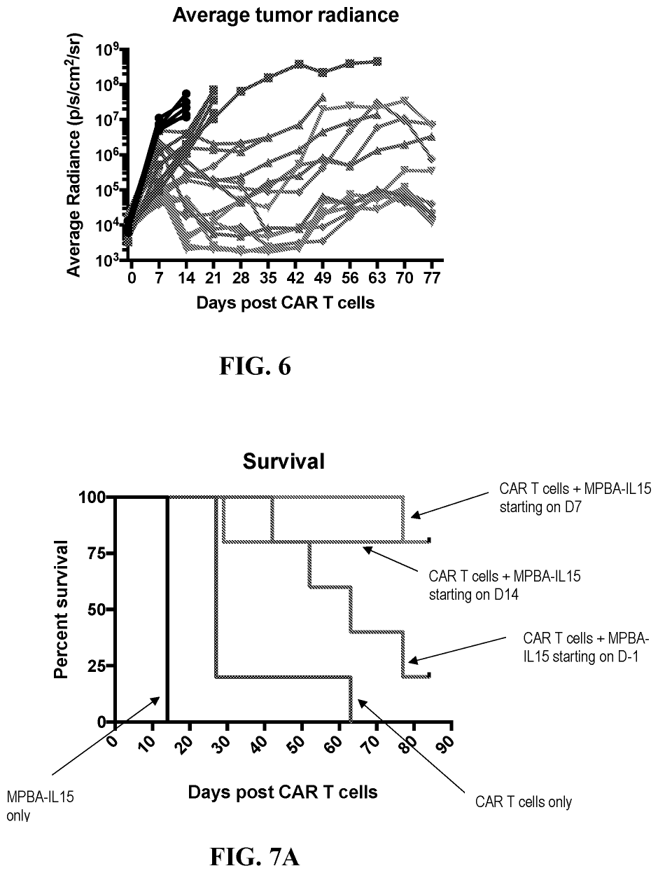

In a further study (Study B), Raji-bearing NSG mice received CAR T cell infusions on DO; 0.30 mg/kg of mono(methoxyPEG-N-butanamide)interleukin-15 was administered on D−1, D7 or D14, and weekly thereafter (5 mice/group). See FIG. 7B. Mice were bled weekly and CD8 and CD4 CAR T cells were identified by flow cytometry (FIGS. 5A and 5B, respectively). Tumor burden was assessed by weekly bioluminescent imaging as shown in FIG. 6 (average tumor radiance versus days post CAR T cell administration) and survival (FIG. 7A).

The results of this preclinical study further indicated that mono(methoxyPEG-N-butanamide)interleukin-15 in combination with CAR T cells results in increased CAR T cells in the blood, decreased tumor burden and increased survival of Raji lymphoma bearing NSG mice.

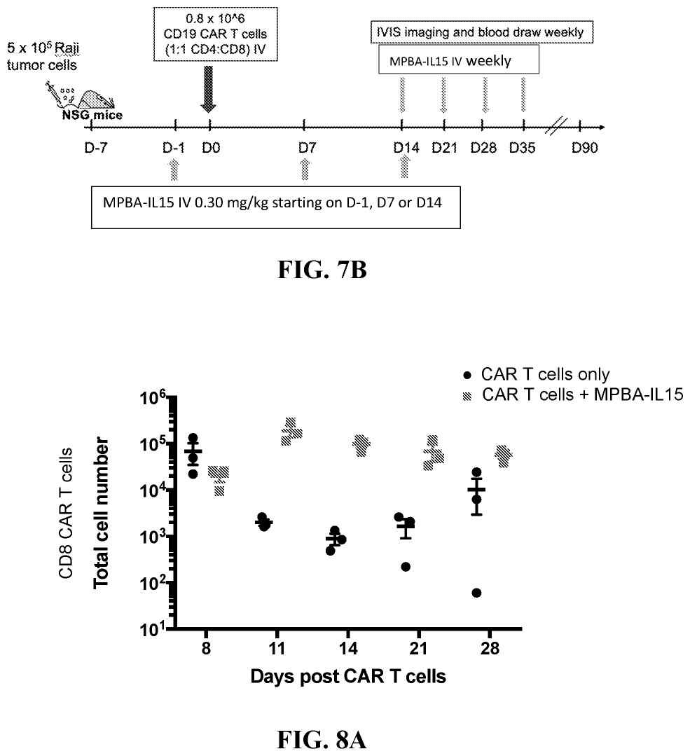

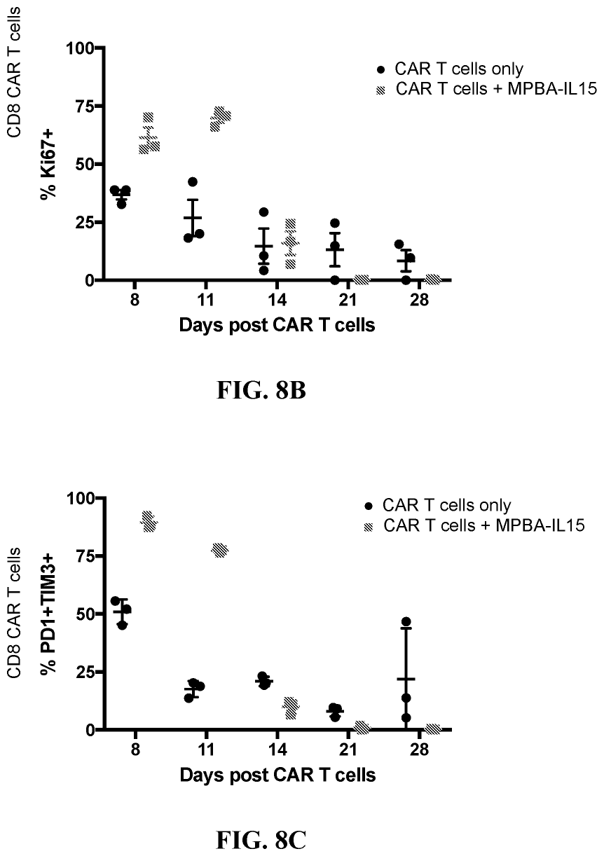

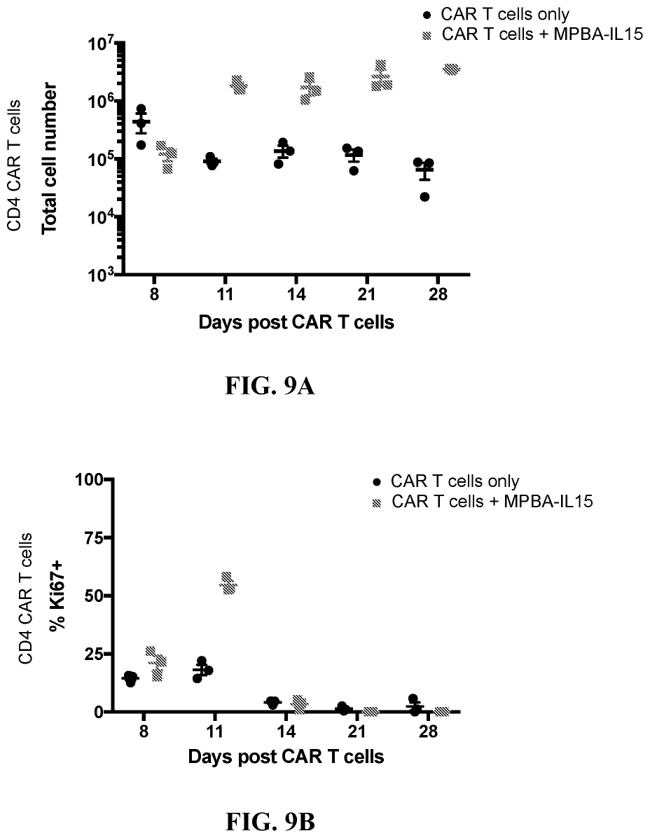

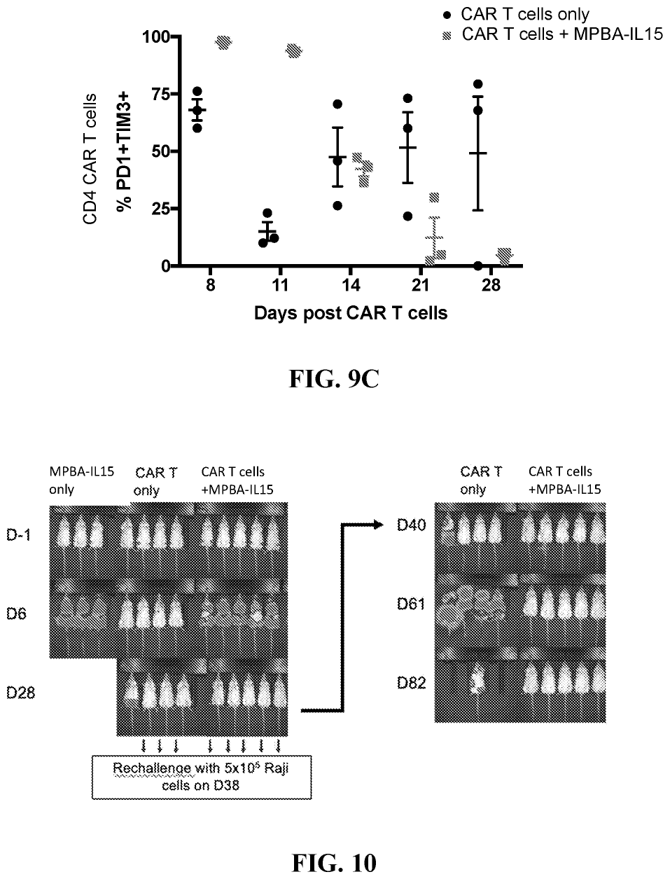

For the mice in the 0.30 mg/kg mono(methoxyPEG-N-butanamide)interleukin-15 dosage group of Study A, mice were euthanized on D8, 11, 14, 21 and 28 post-CAR T cell infusion. Single cell suspensions were made from bone marrow and CAR T cells; total cell numbers (FIGS. 8A, 9A), Ki67 expression (FIGS. 8B, 9B), PD1 and TIM3 expression (FIGS. 8C, 9C) for the CD8 CAR T cell and the CD4 CAR T cell suspensions, respectively, were assessed by flow cytometry. Graphs show mean±SEM.

As shown in FIGS. 8A and 9A, mice treated with the exemplary combination immunotherapy have increased absolute numbers of CAR T cells in the bone marrow. CAR T cell therapy in combination with MPBA-IL-15 resulted in increased accumulation and proliferation of CAR T cells in the bone marrow of Raji-bearing mice and decreased prolonged dual expression of PD1 and TIM3 (FIGS. 8C, 9C).

In vivo, infusion of mono(methoxyPEG-N-butanamide)interleukin-15 starting at D−1, 7 or 14 increased peak CAR T cell numbers in blood. Raji cells were eliminated from marrow by D14 in mice receiving CAR T cells and mono(methoxyPEG-N-butanamide)interleukin-15 on D7 (see in particular the 0.30 mg/kg dosage group), but not in mice receiving CAR T cells alone. Superior mono(methoxyPEG-N-butanamide)interleukin-15 dose-dependent tumor control and survival was observed in mice receiving CAR T cells with mono(methoxyPEG-N-butanamide)interleukin-15 compared to CAR T cells or mono(methoxyPEG-N-butanamide)interleukin-15 alone. Whilst in no way meant to be limiting, the benefit of combination therapy in this preclinical model, based upon this set of experiments, appeared to be greater when administration of mono(methoxyPEG-N-butanamide)interleukin-15 commenced by about D7. Residual CAR T cells in mono(methoxyPEG-N-butanamide)interleukin-15-treated mice rejected re-challenge of Raji tumor cells administered beyond 5 weeks after CAR T cell infusion.

In this lymphoma model, mono(methoxyPEG-N-butanamide)interleukin-15 administration was found to improve the antitumor efficacy and kinetics of administered CD19 CAR T cells. More particularly, exhibiting superior efficacy compared to CAR T cells alone, the combination of CAR T cells and mono(methoxyPEG-N-butanamide)interleukin-15 significantly reduced tumor burden and exerted sustained tumor control, and in some cases, eradicated Raji lymphoma in NSG mice. In contrast, tumor progression was observed in the CAR T cell monotherapy group. See FIG. 4A.

More specifically, 100% of mono(methoxyPEG-N-butanamide)interleukin-15 (0.03 mg/kg)/CAR T cell-treated mice survived 70 days post tumor injection, whereas, in comparison, no mice receiving vehicle control survived to day 14, and no mice treated with CAR T cells alone survived to day 59. Results are shown in FIG. 4A (bioluminescence imaging results) and in FIG. 7A (survival) for mice treated with the 0.30 mg/kg dose of MPBA-IL15. Moreover, mice previously treated with mono(methoxyPEG-N-butanamide)interleukin-15 and CAR T cells were able to reject Raji tumor rechallenge, supporting CAR T cell persistence and potentially long term memory CAR T formation; the striking results are shown in FIG. 10, and illustrate the ability of a long-acting interleukin-15 agonist such as MPBA-IL-15, when administered in combination with CAR-T cell therapy, to not only significantly decrease tumor burden but to also eradicate Raji-lymphoma.

### Example 4

### Investigation of Mono(Methoxypeg-N-Butanamide)Interleukin-15 on Efficacy of ROR1 CAR T Cell Immunotherapy in a Preclinical Murine ROR1 Lung Tumor Model

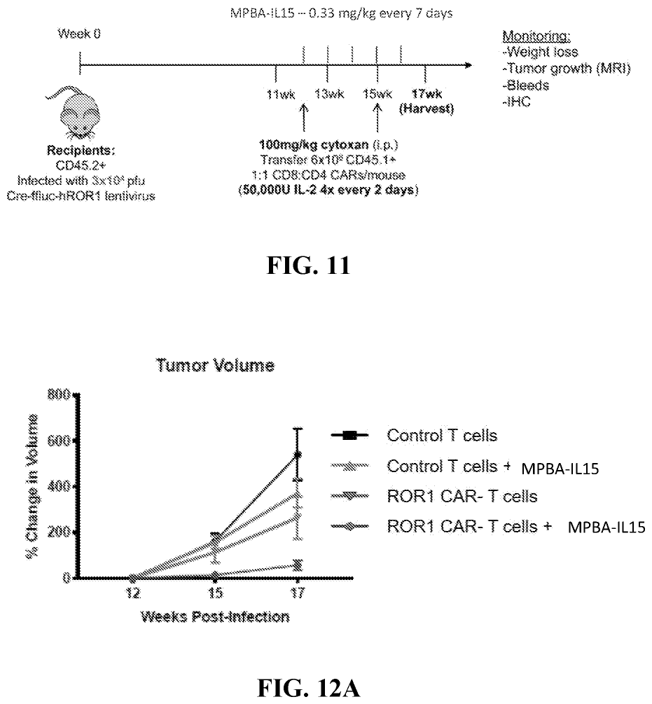

A cohort of Kras^{LSL-G12D/+}p53^{f/f} mice (n=5-6 per group) was infected intratracheally with 3×10⁴ pfu of Cre-flluc-hROR1 lentivirus to induce development of ROR1+ lung tumors. At 12 weeks and 15 weeks post-infection, mice were treated with 100 mg/kg cyclophosphamide for lymphodepletion, and adoptively transferred 6×10⁶ ROR1 CAR T cells or control T cells (1:1 ratio of CD8:CD4) intravenously. ROR1 (receptor tyrosine kinase-like orphan receptor 1) is expressed in numerous malignancies, including a subset of non-small cell lung cancer (NSCLC) and triple negative breast cancer (TNBC). Mice received 5×10⁴ IU IL-2 intraperitoneally every other day for 8 days to support the engraftment of transferred T cells. Beginning on the day of T cell transfer, a subset of mice were treated with 0.33 mg/kg MPBA-IL15 intravenously every 7 days. The preclinical treatment protocol is shown in FIG. 11.

Tumor burden was quantified by acquiring serial 1 mm images spanning the entire lung and summing the tumor area across all images to quantitate tumor volume. At 17 weeks post-infection, all mice were euthanized and whole lungs were analyzed by flow cytometry and immunohistochemistry. To distinguish lung tumor-infiltrating cells from contaminating cells in the circulation, mice were injected intravenously with PE-conjugated anti-CD45 antibody 5 minutes prior to euthanasia to label all immune cells in the circulation, allowing the definition of PE⁻ cells as non-vascular lung parenchymal cells. Lungs were analyzed by IHC staining for CD8a and CD8 infiltration into tumors; staining was quantified using HALO software.

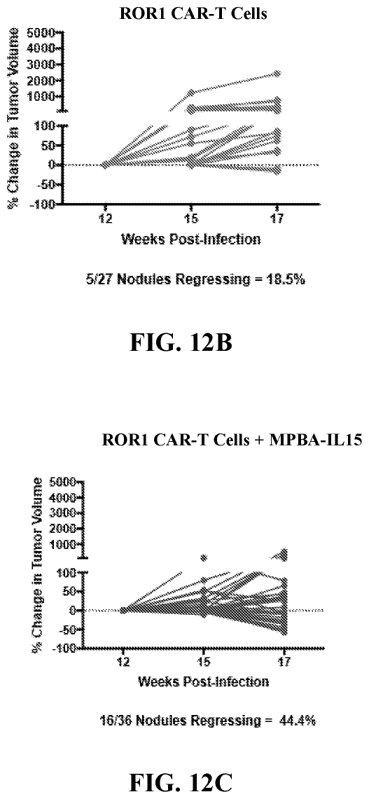

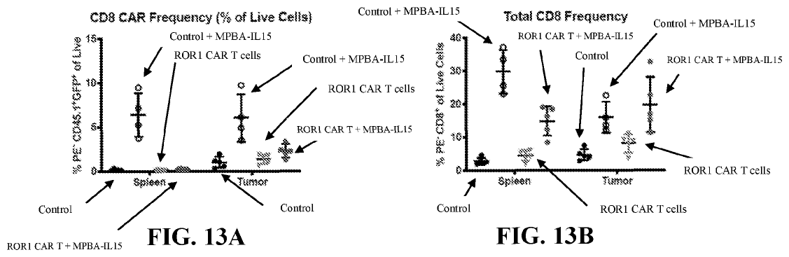

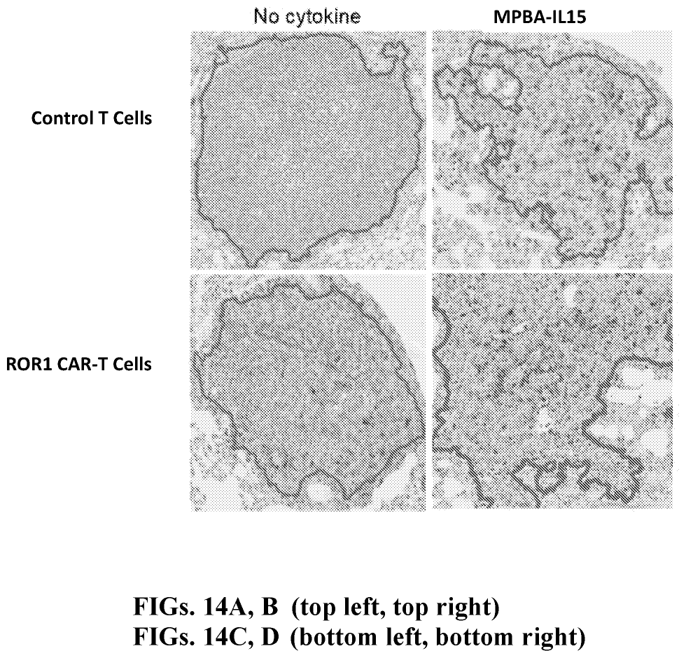

Results are shown in FIG. 12A (% change in tumor volume for mice in the various treatment groups: control T cells (rectangles), control T cells and MPBA-IL15 (▴), ROR1 CAR T cells (▾), and ROR1 CAR T cells and MPBA-IL15 (♦); FIG. 12B (percent change in tumor volume versus weeks post-infection for individual mice treated with ROR1 CAR T cell monotherapy (18.5% regression for group)); FIG. 12C (percent change in tumor volume versus weeks post-infection for individual mice treated with ROR1 CAR T cell and MPBA-IL-15 doublet combination therapy (44.4% regression for group); FIG. 13A (CD8 CAR T cell frequency expressed as a percentage of live cells in the spleen and tumor, respectively, for the various treatment groups), FIG. 13B (CD8 cell frequency expressed as a percentage of live cells in the spleen and tumor, respectively, for the various treatment groups), and FIGS. 14A, B, C, and D (IHC staining illustrating that MPBA-IL-15, an illustrative long acting IL-15 agonist, augments ROR1 CAR T cell trafficking in the lung in this preclinical ROR1 lung cancer model.

The foregoing examples illustrate that administration of MPBA-IL15 significantly improves the antitumor efficacy and kinetics of CD19 CAR T cells in the treatment of a subject having cancer, and augments ROR1 CAR T cell trafficking and persistence in cancerous lung tissue; thus, the instant disclosure provides a new and uniquely advantageous immunotherapy approach for treating a patient with cancer by administering CAR T cell therapy in combination with a long-acting IL-15 agonist such MPBA-IL15.

### Example 5

### CAR-T Cell Numbers and Intracellular Protein Expression in CD8 CAR T Cells Treated with Mono(Methoxypeg-N-Butanamide)Interleukin-15 In Vitro

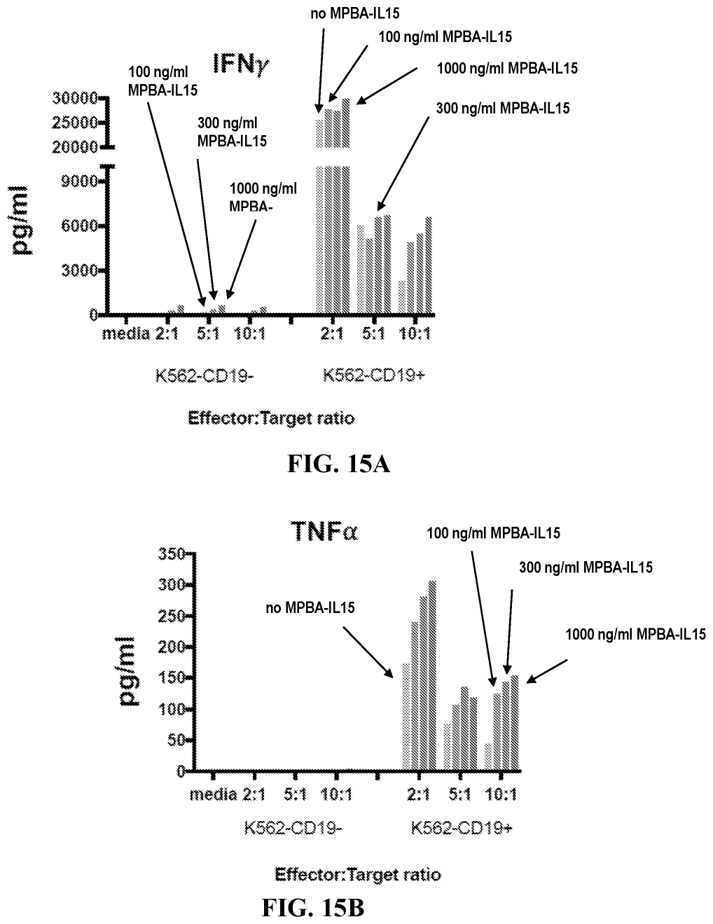

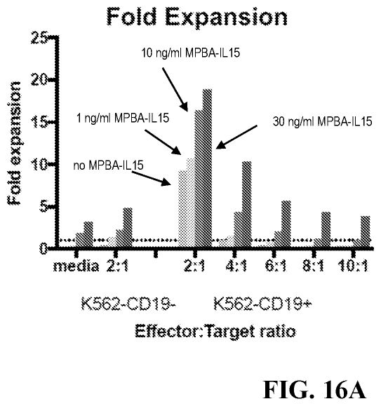

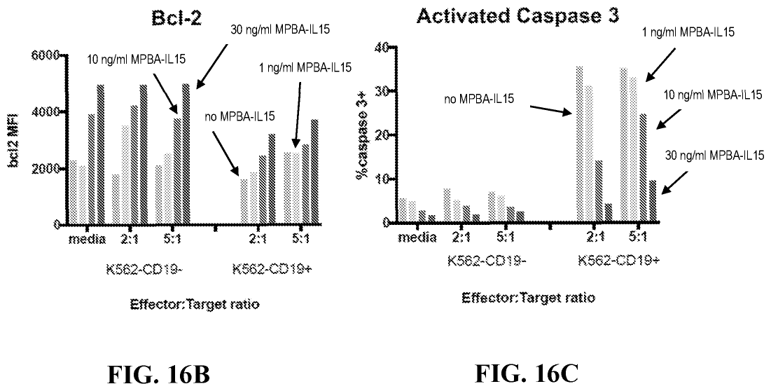

CD8 CAR T cells were generated from healthy donors. On day 15, CAR T cells were co-cultured with irradiated K562-CD19+ or K562-CD19− cells. Cells were either untreated or treated with mono(methoxyPEG-N-butanamide)interleukin-15 (at concentrations of 1 ng/ml, 10 ng/ml, or 30 ng/ml). IFNγ and TNFα production was assayed by Luminex 24 hours after co-culture. CAR T cell numbers and intracellular expression of bcl-2 and activated caspase 3 were determined after 5 days of co-culture. Expression of bcl-2 and activated caspase 3 was determined by flow cytometry. Results are shown in FIGS. 15A, 15B and FIGS. 16A-C.

FIG. 15A provides expression of IFNγ in pg/ml in CD8 CAR T cells co-cultured with irradiated K562-CD19+ or K562-CD19− cells, and either untreated with MPBA-IL15, or treated with MPBA-IL15 at concentrations of 1 ng/ml, 10 ng/ml, or 30 ng/ml.

FIG. 15B provides expression of TNFα in pg/ml in CD8 CAR T cells co-cultured with irradiated K562-CD19+ or K562-CD19− cells, and either untreated with MPBA-IL15, or treated with MPBA-IL15 at concentrations of 1 ng/ml, 10 ng/ml, or 30 ng/ml.

FIG. 16A illustrates CAR T-cell expansion (as fold expansion) for CD8 CAR T cells co-cultured with irradiated K562-CD19+ or K562-CD19− cells, and either untreated with MPBA-IL15, or treated with MPBA-IL15 at concentrations of 1 ng/ml, 10 ng/ml, or 30 ng/ml.

FIGS. 16B and 16C provide expression of bcl-2 (in bcl-2 MFI) and activated caspase 3 (as % caspase 3+), respectively, in CD8 CAR T cells co-cultured with irradiated K562-CD19+ or K562-CD19− cells, and either untreated with MPBA-IL15, or treated with MPBA-IL15 at concentrations of 1 ng/ml, 10 ng/ml, or 30 ng/ml.

From these results, it can be seen that treatment of CAR T cells in vitro with mono(methoxyPEG-N-butanamide)interleukin-15 increases antigen-specific CD8 CAR T cell production and expansion, and enhances survival.

### Example 6

### Protein Expression in CAR T Cells Following Administration of a Combination of CAR T Cells and Mono(Methoxypeg-N-Butanamide)Interleukin-15 in a Preclinical Murine Lymphoma Model

NSG mice received Raji lymphoma cells on D−7, CAR-T cells on DO and weekly injections of mono(methoxyPEG-N-butanamide)interleukin-15 (0.3 mg/kg) starting on D7 and as described in greater detail in Example 3 above. Mice were euthanized on D8, 11, 14, 21 and 28 post CAR T cell infusion. Single cell suspensions were made from bone marrow (2 femur and 2 tibia per mouse). Protein expression (bcl-2, CD45RA and CCR7) was assayed by flow cytometry.

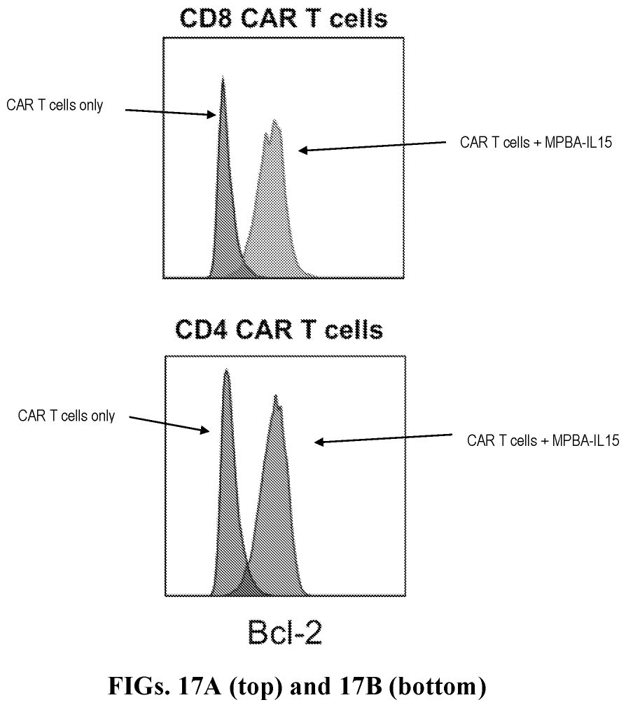

Bcl-2 expression in CAR T cells is shown in histograms as determined on D8 post-infusion (grey=CAR T cell-only mice), red and blue=mice administered CAR T cells and mono(methoxyPEG-N-butanamide)interleukin-15 (MPBA-IL15) for both CD8 CAR T cells (FIG. 17A) and CD4 CAR T cells (FIG. 17B).

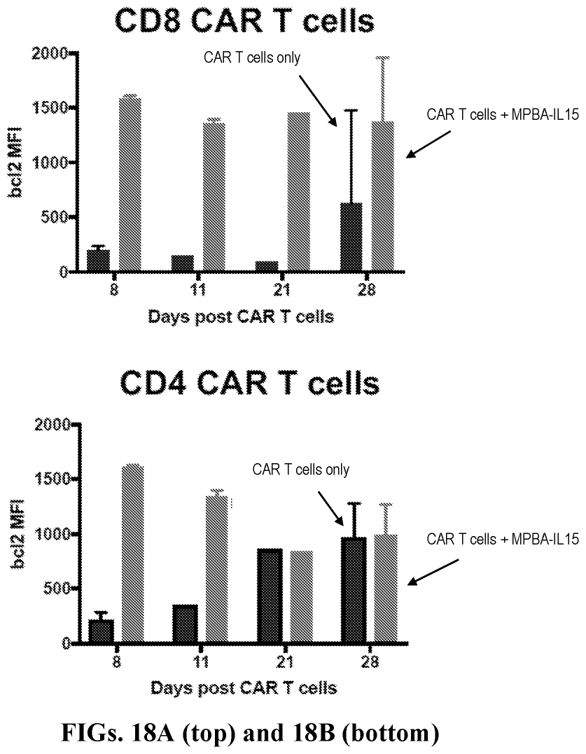

Bcl-2 expression in CAR T cells is also shown in bar graphs as determined on D8 post-infusion (black=CAR T cell-only mice), red=mice administered CAR T cells and mono(methoxyPEG-N-butanamide)interleukin-15 (MPBA-IL15) for both CD8 CAR T cells (FIG. 18A) and CD4 CAR T cells (FIG. 18B).

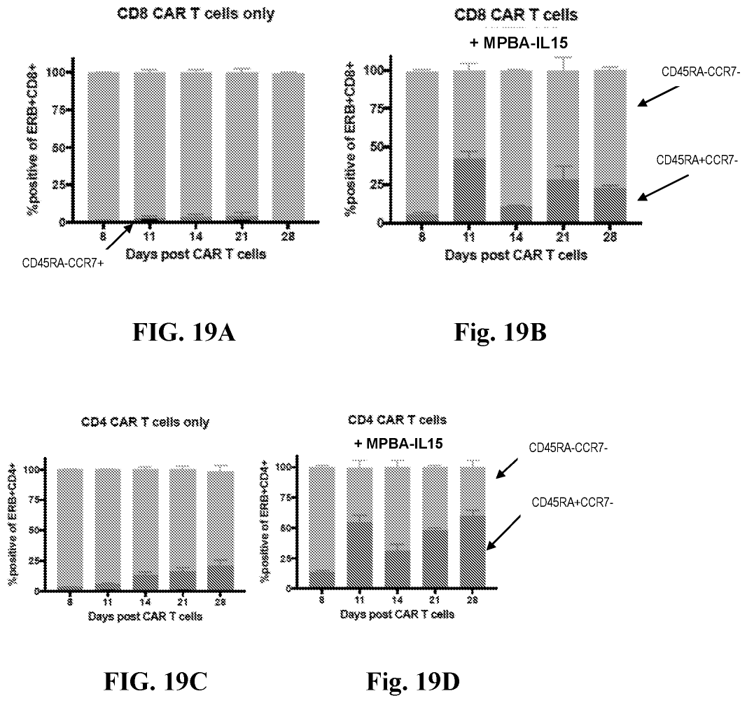

Expression of memory markers CD45RA and CCR7 in CAR T cells is illustrated in FIGS. 19A-19D, where FIG. 19A relates to protein expression in CD8 CAR T cells of mice administered CAR T cells only; FIG. 19B relates to protein expression in CD8 CAR T cells of mice administered CAR T cells and mono(methoxyPEG-N-butanamide)interleukin-15 (MPBA-IL15), FIG. 19C relates to protein expression in CD4 CAR T cells of mice administered CAR T cells only; FIG. 19D relates to protein expression in CD4 CAR T cells of mice administered CAR T cells and mono(methoxyPEG-N-butanamide)interleukin-15 (MPBA-IL15), where expression data is provided for CD5RA−CCR7− (orange), CD5RA+CCR7− (green), and CD5RA-CCR7+ (red). Graphs show mean±SEM.

CAR-T cells treated with mono(methoxyPEG-N-butanamide)interleukin-15 (MPBA-IL15) and CAR-T cells recovered from mice treated with a combination of CAR-T cell therapy and MPBA-IL15 demonstrate increased proliferation and survival both in vitro and in vivo which may in part be due to increased expression of bcl-2.

### Example 7

### Clinical Study

### A Phase 1B/2, Open-Label, Multicenter, Dose Escalation and Dose Expansion Study of Mono(Methoxypeg-N-Butanamide)Interleukin-15 in Combination with CD19-Directed CAR-T Therapy in Patients with B Cell Non-Hodgkin Lymphoma

This is a Phase 1b/2, open-label, multicenter, dose escalation and dose expansion study of mono(methoxyPEG-N-butanamide)interleukin-15 (MPBA-IL15) in combination with CD19+CAR-T in patients with diffuse lymphocytic B cell lymphoma (DLBCL). The study is divided into a screening period, treatment period, end of treatment (eot) period, and long-term follow-up periods.

The MPBA-IL-15 starting dose in dose group 1 will be 1.5 μg/kg. Patients will receive IV MPBA-IL-15 in 21-day cycles, starting on Cycle 1 Day 1.

MPBA-IL-15 drug product is provided as a sterile, white to off-white lyophilized powder. MPBA-IL-15 drug product is formulated in 10 mM potassium phosphate, 260 mM trehalose, 0.02% (w/v) polysorbate 20 at pH 6.8 with approximately 1.0 mg/mL recombinant human IL-15 (rhIL-15). Each vial of MPBA-IL-15 drug product contains 1.1 mg rhIL-15 equivalent. This includes an overfill of 0.1 mg to ensure consistent withdrawal of the label claimed amount of 1.0 mg post reconstitution.

### Duration of Treatment

Dose Escalation (Phase 1b): Patients receiving one of the 2 commercially available CD19 directed chimeric antigen receptor T cell (CD19 CAR-T, Kymriah™ (tisagenlecleucel) or Yescarta® (axicabtagene ciloleucel)) therapy who meet the safety inclusion criteria will receive intravenous (IV) MPBA-IL-15. During dose escalation, MPBA-IL-15 will be given to patients approximately 14 days or 7 days following a single dose of CD19 CAR-T infusion (depending on the cohort assigned). Treatment with MPBA-IL-15 will be up to 8 cycles (6 months) every 21 days (ie, every 3 weeks [q3w]) or until evidence of disease progression, unacceptable toxicity, patient withdrawal, Investigator discretion, or Sponsor decision to terminate the study. Patients who are showing clinical benefit based on the Investigator's judgment may continue treatment with Medical Monitor approval.

Dose Expansion (Phase 2): After determination of the recommended Phase 2 dose (RP2D) of MPBA-IL-15 with either of the CD19 CAR-T products, the RP2D dose will be further investigated in an expansion cohort during Phase 2. Treatment with MPBA-IL-15 will be up to 8 cycles (6 months) every 21 days (ie, every 3 weeks) or until evidence of disease progression, unacceptable toxicity, patient withdrawal, Investigator discretion, or Sponsor decision to terminate the study. Patients who are showing clinical benefit based on the Investigator's judgment may continue treatment with Medical Monitor approval.

### Primary Objectives:

Phase 1b:

(i) To evaluate the safety and tolerability of MPBA-IL-15 after CD19 CAR-T therapy

(ii) To define the maximum tolerated dose(s) (MTD) or RP2D and optimal dosing period of MPBA-IL-15 post CD19 CAR-T administration

Phase 2:

To evaluate the efficacy of MPBA-IL-15 after CD19 CAR-T therapy by assessing the Complete Response Rate (CRR) at 6 months, based on the Lugano Classification (Cheson B D, Fisher R I, Barrington S F, et al. Recommendations for initial evaluation, staging, and response assessment of Hodgkin and non-Hodgkin lymphoma: the Lugano classification. J Clin Oncol. 2014; 32(27):3059.)

### Secondary Objectives:

Phase 1b and Phase 2:

(i) To evaluate Overall Response Rate (ORR) of MPBA-IL-15 in combination with CD19 CAR-T therapy

(ii) To evaluate Progression-free Survival (PFS) of MPBA-IL-15 in combination with CD19 CAR-T therapy

(iii) To evaluate Overall Survival (OS) of MPBA-IL-15 in combination with CD19 CAR-T therapy (Phase 2 only)

(iv) To evaluate Duration of Response (DOR) of MPBA-IL-15 in combination with CD19 CAR-T therapy

(v) To characterize the pharmacokinetics (PK) of NKTR-255 in combination with CD19 CAR-T therapy

(vi) To characterize the pharmacodynamic (PD) effects of NKTR 255 in combination with CD19 CAR-T therapy

(vii) To evaluate PD effects of CD19 CAR-T cells, including duration of in vivo persistence of adoptively transferred T cells and the phenotype of persisting T cells

(viii) To assess immunogenicity of MPBA-IL-15

Exploratory Objectives:

(i) To evaluate Event-free survival (EFS) of MPBA-IL-15 in combination with CD19 CAR-T therapy

(ii) To assess the association between antitumor activity and immune cells in tumor and blood

(iii) To assess trafficking of adoptively transferred T cells to the bone marrow or other tumor site and function in vivo

(iv) To characterize changes in cytokine levels and immune cell populations from baseline

Study Population: Adults aged 18 years and older receiving CD19 CAR-T cells to treat relapsed/refractory (R/R) B cell Non-Hodgkins lymphoma (B-NHL) after 2 or more lines of systemic therapy, including diffuse lymphocytic B cell lymphoma (DLBCL) not otherwise specified, primary mediastinal large B cell lymphoma (PMBCL; Yescarta only), high grade B cell lymphoma, and DLBCL arising from follicular lymphoma.

Number of Patients (Planned):

Phase 1b: Approximately 55 patients will be enrolled

Phase 2: Approximately 60 patients will be enrolled

Number of Study Sites:

Phase 1b: Approximately 5 North America sites

Phase 2: Approximately 5 North America sites

### Study Design:

This study is a Phase 1b/2, open-label, multicenter study consisting of dose escalation (Phase 1b) and dose expansion (Phase 2) portions.

### Phase 1b (Dose Escalation)

Patients receiving US FDA-approved CD19 CAR-T cells (Yescarta or Kymriah) who meet the safety inclusion criteria will receive IV MPBA-IL-15 q3w starting approximately 14 days or 7 days following a single treatment of CD19 CAR-T infusion (depending on the cohort assigned). During dose escalation (Phase 1b), up to 5 cohorts, of at least 3 patients each will be given escalating doses of MPBA-IL-15 after CD19 CAR-T cell infusion. A sample dose escalation schema for MPBA-IL-15 with CD19 CAR-T is provided in the table below. The first patient (a sentinel patient) of each escalating MPBA-IL-15 dose cohort will be monitored for safety and tolerability for 21 days after the first administration of MPBA-IL-15 before additional patients are dosed within the same cohort.

TABLE 4
Sample Dose Levels for MPBA-IL-15 (Phase 1b)*

| MPBA-IL-15 Dose | MPBA-IL-15 | |
|---|---|---|
| Level | Dose | |
| Dose level −2 | 0.375 | ug/kg |
| Dose level −1 | 0.75 | ug/kg |
| Dose level 1 | 1.5 | ug/kg |
| **(starting dose) | | |
| Dose Level 2 | 3 | ug/kg |
| Dose Level 3 | 4.5 | ug/kg |
| Dose Level 4 | 6 | ug/kg |
| Dose Level 5*** | 9 | ug/kg |

q3w: every 3 weeks
*With the exception of the Cohort 1 dose, dose levels for subsequent cohorts may be adjusted based on clinical observations. Dose escalation will not exceed double the prior dose of MPBA-IL-15.
**If dose limiting toxicities are observed at the starting dose, MPBA-IL-15 may be de-escalated as shown in Dose level −1.
***Additional cohort(s) or dose levels may be enrolled following review of safety/PK data and agreement between the Sponsor Medical Monitor and the Principal Investigator.

MPBA-IL-15 will be tested in sequential combination initially starting with Yescarta therapy. The study will initiate with a MPBA-IL-15 starting dose of 1.5 μg/kg IV, administered 14 days following Yescarta infusion (Cohort A). After establishing safety and tolerability in 3 dose levels of MPBA-IL-15 administered 14 days (±3 days) post Yescarta (Cohort A), the next cohorts, Kymriah (14 days post CAR-T infusion; Cohort B) and Yescarta (7 days post CAR-T infusion, Cohort C), will be initiated at the confirmed safe dose level in parallel. The tested dose may be de-escalated, to the dose tested immediately prior, if the 7-day regimen has safety or tolerability issues observed at the starting dose level. The 7-day regimen will be predicated on the observed safety and tolerability of each respective product at the 14-day regimen. Dose escalation will continue exclusively with MPBA-IL-15 with the 7-day regimen until the MTD or RP2D is established. If the 7-day regimen is found to not be tolerated, then MPBA-IL-15 dose escalation in the 14-day regimen may resume.

A 2-parameter Bayesian logistic regression model (BLRM) employing the escalation with overdose control (EWOC) principle (Neuenschwander B, Branson M, Gsponer T. Critical aspects of the Bayesian approach to phase I cancer trials. Stat Med. 2008 Jun. 15; 27(13):2420-39) will be used during the escalation phase of the study for dose level selection and for determination of the MTD. The MTD will be declared when at least 6 patients have been evaluated at a dose and the posterior probability of targeted toxicity is at least 70% for that dose. The MTD will be determined based on criteria summarized in Section 5.8. Additional cohorts may also be opened to further explore the MTD.

The RP2D of MPBA-IL-15 in combination with CD19 CAR-T will be chosen at a dose not exceeding the final recommendation from dose escalation and will be based upon review of all available data for safety, PK, PD, and an optimal biological response of MPBA-IL-15. Additional patients may be enrolled to refine the RP2D, and a minimum of 6 patients (including any patients from dose escalation) dosed at the chosen RP2D will be required to determine the RP2D.

Additional rules regarding dose escalation during Phase 1b are below:

No intra-patient dose escalation will be allowed.

Enrollment into a new cohort with an escalating dose of MPBA-IL-15 cannot begin until the dose limiting toxicity (DLT) window has elapsed since the last patient's first MPBA-IL-15 administration in the previous cohort. The DLT window is 21 days following MPBA-IL-15 administration.

Escalation to a higher dose will occur only if there is experience with that dose in the FIH study (MPBA-IL-15-002).

For dose escalation cohorts, the Sponsor medical monitor and Safety Review Committee (SRC) will jointly assess safety before opening dose escalation to the next cohort.

Dose levels of MPBA-IL-15 for a given cohort may be reduced depending on the severity, duration, and frequency of toxicities observed at the previous dose level tested.

The decision to declare the RP2D of MPBA-IL-15 subsequent to CD19 CAR-T infusion can occur at any given dose level or starting day based on safety, PK, PD or an optimal biological response without reaching the MTD.

### Phase 2 (Dose Expansion)

In Phase 2 of the study, enrollment into the dose expansion cohort will commence once the RP2D is established for each respective CD19 CAR-T product. The decision for selection of the specific CD19 CAR-T product and schedule for Phase 2 will be based upon review of all available data on safety, PK, PD, and an optimal biological response of MPBA-IL-15 subsequent to CAR-T infusion in Phase 1b. Patients will receive IV MPBA-IL-15 at the RP2D and schedule determined in Phase 1b either 14 or 7 days after CD19 CAR-T infusion. Treatment with MPBA-IL-15 will be repeated every 21 days (ie, every 3 weeks) for up to 8 cycles (6 months).

### Key Eligibility Criteria

Eligibility will be determined within 1 month before leukapheresis for CD19 CAR-T cell manufacturing. If intensive bridging chemotherapy or radiation therapy is administered, eligibility criteria should be re-evaluated before lymphodepletion.
Male or female patients, 218 years of age, on the day of signing the informed consent form (ICF)
Eligible for commercial CD19 CAR-T cell therapy
Confirmed diagnosis of B-NHL, including DLBCL not otherwise specified, PMBCL (Yescarta only), high grade B cell lymphoma; and DLBCL arising from follicular lymphoma
Relapsed or refractory (R/R) disease, defined as detectable disease after 2 or more lines of therapy, including an anthracycline and either having failed autologous hematopoietic stem cell transplantation (ASCT), or being ineligible for or not consenting to ASCT
Measurable fluorodeoxyglucose (FDG) avid nodal and/or extranodal disease by Lugano Classification (Cheson, 2014, ibid) that can be accurately measured in at least 1 dimension as ≥1.5 cm
Life expectancy >30 days
Acceptable organ function, as defined as:
   Adequate pulmonary function, defined as Grade: 1 dyspnea and oxygen saturation ≥92% on room air. If these parameters are not met, at the discretion of the treating physician, patients with FEV1≥ 50% of predicted and diffusing capacity of the lungs for carbon monoxide (DLCO; corrected) of ≥40% of predicted on pulmonary function testing (PFT) will be eligible.
   Adequate cardiac function, defined as left ventricular ejection fraction (LVEF)≥45%, or LVEF 40-44% and clearance by a cardiologist.
   Adequate renal function defined as:
      A serum creatinine of ≤1.5× upper limit of normal (ULN) OR, eGFR≥60 mL/min/1.73 m²
   Adequate liver function defined as:
      Aspartate aminotransferase (AST) and alanine aminotransferase (ALT)≤3×ULN
      Bilirubin ≤2.0 mg/dL with the exception of patients with Gilbert-Meulengracht syndrome; patients with Gilbert-Meulengracht syndrome may be included if their total bilirubin is ≤3.0×ULN and direct bilirubin ≤1.5×ULN
   Adequate bone marrow reserve without transfusions defined as:
      Absolute neutrophil count (ANC)>1000/mm³
      Absolute lymphocyte count (ALC)≥300/mm³
      Platelets ≥50,000/mm³
         Hemoglobin >8.0 g/dL

### Safety Eligibility Assessed Prior to the Initial and Subsequent MPBA-IL-15 Doses

Patients will be eligible for MPBA-IL-15 infusion if they fulfill the following criteria:
1. Received a CD19 CAR-T infusion
2. No persisting Grade ≥1 cytokine release syndrome (CRS) (temperature ≥38.0° C.) on the day of MPBA-IL-15 infusion
3. No Grade 4 CRS within 96 hours preceding MPBA-IL-15 infusion
4. No persisting Grade ≥2 neurotoxicity on the day of MPBA-IL-15 infusion
5. No previous Grade ≥3 neurotoxicity of ≥48 hours duration at any time preceding MPBA-IL-15 infusion
6. No intervention with tocilizumab and/or dexamethasone within 48 hours preceding MPBA-IL-15 infusion
7. No active, serious, and uncontrolled infection(s)
8. No contraindications according to the Investigator's assessment
9. Patient has adequate organ function prior to all doses of MPBA-IL-15:
   a) AST and ALT level ≤3×ULN;
   b) Total bilirubin level ≤3×ULN
   c) eGFR>30 mL/min
   d) DLCO>40%
   e) LVEF>45%

### Test Product, Dose and Mode of Administration

Reconstituted MPBA-IL-15 will be administered IV every 21 days (ie, q3w). Reconstituted MPBA-IL-15 is to be further diluted with commercially available or 0.9% normal saline solutions for injection. The final diluted solution will be infused over 30±5 minutes.

The MPBA-IL-15 starting dose will be 1.5 μg/kg every 21 days.

### Safety

Assessment of safety will occur by ongoing review of the following:
incidence of adverse events (AEs), including serious AEs (SAEs), and immune-mediated AEs (imAEs)
clinical laboratory tests (blood and urine sampling)
vital signs
electrocardiograms (ECG)
physical examination
concomitant medication
   DLTs—Phase 1b only

### Pharmacokinetics

Blood samples for MPBA-IL-15 PK analyses will be collected from all patients. Serial PK samples will be collected at multiple scheduled sampling times after each cycle of MPBA-IL-15. Plasma concentrations of MPBA-IL-15 will be measured for each PK sample using validated method(s). Pharmacokinetic parameters such as maximum concentration (Cₘₐₓ), area under the concentration-time curve (AUC), clearance (CL), volume of distribution (Vd), and half-life (t1/2) will be estimated from plasma concentration-time data where possible.

### Biomarkers

Systemic and tumor tissue-based (blood and bone marrow) PD effects of MPBA-IL-15 in combination with CD19 CAR-T will be examined.

Characterization as well as monitoring of the genetically modified CD19 CAR-T cells will be performed in peripheral blood as well as bone marrow samples by quantitative polymerase chain reaction (qPCR) and flow cytometry before and during treatment with NKTR 255. Blood samples for systemic PD analyses will be collected before and during treatment from all patients to assess the effects of MPBA-IL-15 on the number and activation of immune cell populations, including, but not limited to, NK cells, CD8+ T cells, and CD8+ memory cells. Blood samples also will be collected before and during treatment to determine changes in cytokine levels and to profile changes in gene expression in response to MPBA-IL-15.

Fresh bone marrow biopsies before, during, and after treatment according to the Schedule of Events will be collected where feasible for characterization of tumor cells and immune system activation. Assessments will include evaluation of changes in tumor-specific protein markers and immune cell populations in the tumor microenvironment. Archival tumor tissue samples if available will be collected and may be analyzed in the same way.

### Efficacy:

18F-FDG− positron emission tomography (PET)/computed tomography (CT) whole body (base of skull through mid-thighs) will be done during screening for baseline assessment and to establish eligibility for measurable disease (SUVₘₐₓ). Follow-up FDG-PET/CT for efficacy assessment per Lugano Classification (Cheson, 2014, ibid) will be performed at Week 4, Month 3 (immediately prior to Cycle 5), and then every 12 weeks until the subject discontinues the study, as well as at the time of disease progression if feasible. Tumor biopsy if feasible, will be obtained approximately at baseline, in the first 4 weeks after CD19 CAR-T infusion, and Week 14; and at the Investigator's discretion at end of treatment (EOT).

### Statistical Methods:

### Safety:

Safety assessments will include AEs, clinical laboratory tests, vital signs, physical examinations, and ECGs (central read). The incidence of DLTs will be evaluated for each dose escalation cohort. All Grade ≥3 treatment-emergent adverse events (TEAEs) will be summarized by system organ class and preferred term for each dose cohort separately in the Phase 1b and Phase 2 of the study. TEAEs will be summarized by incidence, severity, and relationship to study drug(s). Immune-mediated AEs (imAEs) will be summarized separately.

Grade ≥3 clinical laboratory and vital sign abnormalities will be summarized descriptively for each dose cohort in Phase 1b and Phase 2 of the study.

### Efficacy:

Efficacy assessments of CRR at 6 months and ORR will be calculated along with the 95% confidence intervals (CIs) based on exact method. The Kaplan-Meier method will be used for the analyses of PFS, DOR and OS. CRR at 6 months based on an independent review committee (IRC) assessment will be the primary efficacy endpoint and will be summarized using the modified intent-to-treat population. CRR at 6 months based on the investigator's assessment will also be assessed.

The primary analysis for all efficacy endpoints will be based upon patients from the dose expansion part of the study as well as patients who are treated at the RP2D from dose escalation part of the study.

### Pharmacokinetic and Biomarkers:

Pharmacokinetic parameters will be tabulated and summarized with descriptive statistics. Descriptive summaries for biomarkers will be calculated at each observation time. Changes in biomarkers from pre-dose to each observation time will also be assessed using descriptive summaries.

1. A method for treating a subject having cancer, comprising:
(i) administering to the subject an adoptive cellular immunotherapy composition comprising T cells that have been modified to express a chimeric antigen receptor (CAR-T cells); and
(ii) administering to the subject an IL-15 receptor agonist having a structure: 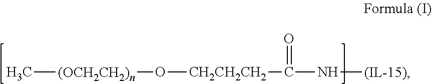
wherein IL-15 is an interleukin-15 moiety, (n) is an integer from about 150 to about 3,000, and ˜NH˜ represents an amino group of the IL-15 moiety.

2. The method of claim 1, wherein the adoptive cellular immunotherapy composition comprises CAR T cells that have been modified to express a CD19-directed chimeric antigen receptor.

3. The method of claim 1, wherein step (i) and step (ii) are carried out sequentially, in either order, or substantially simultaneously.

4. The method of claim 1, wherein step (i) is carried out before step (ii).

5. The method of claim 1, wherein step (ii) is carried out before step (i).

6. The method of claim 1, wherein both steps (i) and (ii) are carried out substantially simultaneously.

7. The method of claim 1, wherein steps (i) and (ii) are both carried out on the same day.

8. The method of claim 4, wherein step (ii) is carried out on (a) any one of days 1 to 7 following step (i), or (b) is carried out on any one of days 8 to 14 following step (i), or (c) is carried out on day 7 or on day 14 following step (i).

9. The method of claim 1, comprising a single administration to the subject of the adoptive cellular immunotherapy composition comprising T cells that have been modified to express a chimeric antigen receptor over the course of treatment.

10. The method of claim 1, comprising multiple administrations to the subject of the IL-15 receptor agonist over the course of treatment.

11. The method of claim 1, wherein the adoptive cellular immunotherapy composition is administered by infusion and/or the IL-15 receptor agonist is administered by infusion.

12. The method of claim 1, wherein the subject is human.

13. The method of claim 1, wherein the cancer is a blood cancer.

14. The method of claim 1, wherein the cancer is a solid tumor.

15. The method of claim 13, wherein the cancer is lymphoma or leukemia.

16. The method of claim 15, wherein the cancer is a B-cell lymphoma.

17. The method of claim 1, wherein the IL-15 receptor agonist has a structure of Formula (I) wherein (n) is in a range from about 795 to about 1068.

18. The method of claim 17, wherein (n) is in a range from about 840 to about 1023.

19. The method of claim 17, wherein on average, (n) has a value of about 907 corresponding to the poly(ethylene) glycol portion of the molecule having a weight average molecular weight of about 40,000 daltons).

20. The method of claim 1, wherein the method is effective to provide a beneficial response to treatment that is enhanced over the response to treatment observed when administration is carried out according to either step (i) or step (ii) alone.

21. The method of claim 20, wherein the beneficial response to treatment is based on a suitable animal model.

22. The method of claim 21, wherein the model is an in vivo xenogenetic B cell lymphoma model.

23. The method of claim 21, wherein the beneficial response to treatment is selected from percent change in tumor volume and total number of CAR-T cells in the bone marrow or tumor tissue, as evaluated at day 45 following tumor cell injection.

24. The method of claim 1, wherein the adoptive cellular immunotherapy composition comprises CD-19-directed genetically modified autologous T cells.

25. The method of claim 1, comprising in step (i) administering to the subject from about 10⁷ to about 10⁹ cells/kg CAR-T cells.

26. The method of claim 25, comprising administering to the subject an amount of CAR-T cells selected from about 0.2×10⁶ cells/kg to about 6.0×10⁸ cells/kg, at least about 2.0×10⁶ cells/kg to about 2.0×10⁸ cells/kg, at least about 0.2×10⁶ cells/kg to about 5.0×10⁶ cells/kg, at least about 0.1×10⁸ cells/kg to about 2.5×10⁸ cells/kg, or at least about 0.6×10⁸ cells/kg to about 6.0×10⁸ cells/kg.

27. The method of claim 1, comprising administering to the subject from about 0.10-50 μg/kg of the IL-15 receptor agonist.

28. The method of claim 27, comprising administering to the subject from about 0.25-25 μg/kg of the IL-15 receptor agonist.

29. The method of claim 28, comprising administering to the subject from about 0.25-15 μg/kg of the IL-15 receptor agonist.

30. The method of claim 1, comprising in step (i), a single administration the adoptive cellular immunotherapy composition, and in step (ii), an initial administration of the IL-15 receptor agonist on any one of days 1 to 14 following step (i), followed by administration of the IL-15 receptor agonist every 21 days over the course of treatment.

31. The method of claim 30, wherein the course of treatment comprises from 1 to 24 cycles of administering of the IL-15 receptor agonist, or from 3 to 20 cycles of administering the IL-15 receptor agonist, or from 4 to 15 cycles of administering the IL-15 receptor agonist.

32. The method of claim 20, effective to thereby provide a response to treatment that is enhanced over the response of a subject to treatment that comprises administration of the adoptive cellular immunotherapy composition alone in absence of administration of the IL-15 receptor agonist.
